SEQUENCE LISTING (12 sequences)

SEQ ID NO: 1 - DNA, 6267 bp, Homo sapiens
agctgcaagtggcgggcgcccaggcagatgcgatccagcggctctgggggcggcagcggtggtagcagctggtacctcccgccgcctctgttcggagggtcgcggggcaccgaggtgctttccggccgccctctggtcggccacccaaagccgcgggcgctgatgatgggtgaggagggggcggcaagatttcgggcgcccctgccctgaacgccctcagctgctgccgccggggccgctccagtgcctgcgaactctgaggagccgaggcgccggtgagagcaaggacgctgcaaacttgcgcagcgcgggggctgggattcacgcccagaagttcagcaggcagacagtccgaagccttcccgcagcggagagatagcttgagggtgcgcaagacggcagcctccgccctcggttcccgcccagaccgggcagaagagcttggaggagccaaaaggaacgcaaaaggcggccaggacagcgtgcagcagctgggagccgccgttctcagccttaaaagttgcagagattggaggctgccccgagaggggacagaccccagctccgactgcggggggcaggagaggacggtacccaactgccacctcccttcaaccatagtagttcctctgtaccgagcgcagcgagctacagacgggggcgcggcactcggcgcggagagcgggaggctcaaggtcccagccagtgagcccagtgtgcttgagtgtctctggactcgcccctgagcttccaggtctgtttcatttagactcctgctcgcctccgtgcagttgggggaaagcaagagacttgcgcgcacgcacagtcctctggagatcaggtggaaggagccgctgggtaccaaggactgttcagagcctcttcccatctcggggagagcgaagggtgaggctgggcccggagagcagtgtaaacggcctcctccggcgggatgggagccatcgggctcctgtggctcctgccgctgctgctttccacggcagctgtgggctccgggatggggaccggccagcgcgcgggctccccagctgcggggccgccgctgcagccccgggagccactcagctactcgcgcctgcagaggaagagtctggcagttgacttcgtggtgccctcgctcttccgtgtctacgcccgggacctactgctgccaccatcctcctcggagctgaaggctggcaggcccgaggcccgcggctcgctagctctggactgcgccccgctgctcaggttgctggggccggcgccgggggtctcctggaccgccggttcaccagccccggcagaggcccggacgctgtccagggtgctgaagggcggctccgtgcgcaagctccggcgtgccaagcagttggtgctggagctgggcgaggaggcgatcttggagggttgcgtcgggccccccggggaggcggctgtggggctgctccagttcaatctcagcgagctgttcagttggtggattcgccaaggcgaagggcgactgaggatccgcctgatgcccgagaagaaggcgtcggaagtgggcagagagggaaggctgtccgcggcaattcgcgcctcccagccccgccttctcttccagatcttcgggactggtcatagctccttggaatcaccaacaaacatgccttctccttctcctgattattttacatggaatctcacctggataatgaaagactccttccctttcctgtctcatcgcagccgatatggtctggagtgcagctttgacttcccctgtgagctggagtattcccctccactgcatgacctcaggaaccagagctggtcctggcgccgcatcccctccgaggaggcctcccagatggacttgctggatgggcctggggcagagcgttctaaggagatgcccagaggctcctttctccttctcaacacctcagctgactccaagcacaccatcctgagtccgtggatgaggagcagcagtgagcactgcacactggccgtctcggtgcacaggcacctgcagccctctggaaggtacattgcccagctgctgccccacaacgaggctgcaagagagatcctcctgatgcccactccagggaagcatggttggacagtgctccagggaagaatcgggcgtccagacaacccatttcgagtggccctggaatacatctccagtggaaaccgcagcttgtctgcagtggacttctttgccctgaagaactgcagtgaaggaacatccccaggctccaagatggccctgcagagctccttcacttgttggaatgggacagtcctccagcttgggcaggcctgtgacttccaccaggactgtgcccagggagaagatgagagccagatgtgccggaaactgcctgtgggtttttactgcaactttgaagatggcttctgtggctggacccaaggcacactgtcaccccacactcctcaatggcaggtcaggaccctaaaggatgcccggttccaggaccaccaagaccatgctctattgctcagtaccactgatgtccccgcttctgaaagtgctacagtgaccagtgctacgtttcctgcaccgatcaagagctctccatgtgagctccgaatgtcctggctcattcgtggagtcttgaggggaaacgtgtccttggtgctagtggagaacaaaaccgggaaggagcaaggcaggatggtctggcatgtcgccgcctatgaaggcttgagcctgtggcagtggatggtgttgcctctcctcgatgtgtctgacaggttctggctgcagatggtcgcatggtggggacaaggatccagagccatcgtggcttttgacaatatctccatcagcctggactgctacctcaccattagcggagaggacaagatcctgcagaatacagcacccaaatcaagaaacctgtttgagagaaacccaaacaaggagctgaaacccggggaaaattcaccaagacagacccccatctttgaccctacagttcattggctgttcaccacatgtggggccagcgggccccatggccccacccaggcacagtgcaacaacgcctaccagaactccaacctgagcgtggaggtggggagcgagggccccctgaaaggcatccagatctggaaggtgccagccaccgacacctacagcatctcgggctacggagctgctggcgggaaaggcgggaagaacaccatgatgcggtcccacggcgtgtctgtgctgggcatcttcaacctggagaaggatgacatgctgtacatcctggttgggcagcagggagaggacgcctgccccagtacaaaccagttaatccagaaagtctgcattggagagaacaatgtgatagaagaagaaatccgtgtgaacagaagcgtgcatgagtgggcaggaggcggaggaggagggggtggagccacctacgtatttaagatgaaggatggagtgccggtgcccctgatcattgcagccggaggtggtggcagggcctacggggccaagacagacacgttccacccagagagactggagaataactcctcggttctagggctaaacggcaattccggagccgcaggtggtggaggtggctggaatgataacacttccttgctctgggccggaaaatctttgcaggagggtgccaccggaggacattcctgcccccaggccatgaagaagtgggggtgggagacaagagggggtttcggagggggtggaggggggtgctcctcaggtggaggaggcggaggatatataggcggcaatgcagcctcaaacaatgaccccgaaatggatggggaagatggggtttccttcatcagtccactgggcatcctgtacaccccagctttaaaagtgatggaaggccacggggaagtgaatattaagcattatctaaactgcagtcactgtgaggtagacgaatgtcacatggaccctgaaagccacaaggtcatctgcttctgtgaccacgggacggtgctggctgaggatggcgtctcctgcattgtgtcacccaccccggagccacacctgccactctcgctgatcctctctgtggtgacctctgccctcgtggccgccctggtcctggctttctccggcatcatgattgtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagagatctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactcacttctcttccttgggatccctaagaccgtggaggagagagaggcaatggctccttcacaaaccagagaccaaatgtcacgttttgttttgtgccaacctattttgaagtaccaccaaaaaagctgtattttgaaaatgctttagaaaggttttgagcatgggttcatcctattctttcgaaagaagaaaatatcataaaaatgagtgataaatacaaggcccagatgtggttgcataaggtttttatgcatgtttgttgtatacttccttatgcttctttcaaattgtgtgtgctctgcttcaatgtagtcagaattagctgcttctatgtttcatagttggggtcatagatgtttccttgccttgttgatgtggacatgagccatttgaggggagagggaacggaaataaaggagttatttgtaatgactaaaa

SEQ ID NO: 2 - DNA, 7368 bp, Homo sapiens
caagctttcaagcattcaaaggtctaaatgaaaaaggctaagtattatttcaaaaggcaagtatatcctaatatagcaaaacaaacaaagcaaaatccatcagctactcctccaattgaagtgatgaagcccaaataattcatatagcaaaatggagaaaattagaccggccatctaaaaatctgccattggtgaagtgatgaagaacatttactgtcttattccgaagcttgtcaattttgcaactcttggctgcctatggatttctgtggtgcagtgtacagttttaaatagctgcctaaagtcgtgtgtaactaatctgggccagcagcttgaccttggcacaccacataatctgagtgaaccgtgtatccaaggatgtcacttttggaactctgtagatcagaaaaactgtgctttaaagtgtcgggagtcgtgtgaggttggctgtagcagcgcggaaggtgcatatgaagaggaagtactggaaaatgcagacctaccaactgctccctttgcttcttccattggaagccacaatatgacattacgatggaaatctgcaaacttctctggagtaaaatacatcattcagtggaaatatgcacaacttctgggaagctggacttatactaagactgtgtccagaccgtcctatgtggtcaagcccctgcaccccttcactgagtacattttccgagtggtttggatcttcacagcgcagctgcagctctactcccctccaagtcccagttacaggactcatcctcatggagttcctgaaactgcacctttgattaggaatattgagagctcaagtcccgacactgtggaagtcagctgggatccacctcaattcccaggtggacctattttgggttataacttaaggctgatcagcaaaaatcaaaaattagatgcagggacacagagaaccagtttccagttttactccactttaccaaatactatctacaggttttctattgcagcagtaaatgaagttggtgagggtccagaagcagaatctagtattaccacttcatcttcagcagttcaacaagaggaacagtggctctttttatccagaaaaacttctctaagaaagagatctttaaaacatttagtagatgaagcacattgccttcggttggatgctatataccataatattacaggaatatctgttgatgtccaccagcaaattgtttatttctctgaaggaactctcatatgggcgaagaaggctgccaacatgtctgatgtatctgacctgagaattttttacagaggttcaggattaatttcttctatctccatagattggctttatcaaagaatgtatttcatcatggatgaactggtatgtgtctgtgatttagagaactgctcaaacatcgaggaaattactccaccctctattagtgcacctcaaaaaattgtggctgattcatacaatgggtatgtcttttacctcctgagagatggcatttatagagcagaccttcctgtaccatctggccggtgtgcagaagctgtgcgtattgtggagagttgcacgttaaaggactttgcaatcaagccacaagccaagcgaatcatttacttcaatgacactgcccaagtcttcatgtcaacatttctggatggctctgcttcccatctcatcctacctcgcatcccctttgctgatgtgaaaagttttgcttgtgaaaacaatgactttcttgtcacagatggcaaggtcattttccaacaggatgctttgtcttttaatgaattcatcgtgggatgtgacctgagtcacatagaagaatttgggtttggtaacttggtcatctttggctcatcctcccagctgcaccctctgccaggccgcccgcaggagctttcggtgctgtttggctctcaccaggctcttgttcaatggaagcctcctgcccttgccataggagccaatgtcatcctgatcagtgatattattgaactctttgaattaggcccttctgcctggcagaactggacctatgaggtgaaagtatccacccaagaccctcctgaagtcactcatattttcttgaacataagtggaaccatgctgaatgtacctgagctgcagagtgctatgaaatacaaggtttctgtgagagcaagttctccaaagaggccaggcccctggtcagagccctcagtgggtactaccctggtgccagctagtgaaccaccatttatcatggctgtgaaagaagatgggctttggagtaaaccattaaatagctttggcccaggagagttcttatcctctgatataggaaatgtgtcagacatggattggtataacaacagcctctactacagtgacacgaaaggcgacgtttttgtgtggctgctgaatgggacggatatctcagagaattatcacctacccagcattgcaggagcaggggctttagcttttgagtggctgggtcactttctctactgggctggaaagacatatgtgatacaaaggcagtctgtgttgacgggacacacagacattgttacccacgtgaagctattggtgaatgacatggtggtggattcagttggtggatatctctactggaccacactctattcagtggaaagcaccagactaaatggggaaagttcccttgtactacagacacagccttggttttctgggaaaaaggtaattgctctaactttagacctcagtgatgggctcctgtattggttggttcaagacagtcaatgtattcacctgtacacagctgttcttcggggacagagcactggggataccaccatcacagaatttgcagcctggagtacttctgaaatttcccagaatgcactgatgtactatagtggtcggctgttctggatcaatggctttaggattatcacaactcaagaaataggtcagaaaaccagtgtctctgttttggaaccagccagatttaatcagttcacaattattcagacatcccttaagcccctgccagggaacttttcctttacccctaaggttattccagattctgttcaagagtcttcatttaggattgaaggaaatgcttcaagttttcaaatcctgtggaatggtccccctgcggtagactggggtgtagttttctacagtgtagaatttagtgctcattctaagttcttggctagtgaacaacactctttacctgtatttactgtggaaggactggaaccttatgccttatttaatctttctgtcactccttatacctactggggaaagggccccaaaacatctctgtcacttcgagcacctgaaacagttccatcagcaccagagaaccccagaatatttatattaccaagtggaaaatgctgcaacaagaatgaagttgtggtggaatttaggtggaacaaacctaagcatgaaaatggggtgttaacaaaatttgaaattttctacaatatatccaatcaaagtattacaaacaaaacatgtgaagactggattgctgtcaatgtcactccctcagtgatgtcttttcaacttgaaggcatgagtcccagatgctttattgccttccaggttagggcctttacatctaaggggccaggaccatatgctgacgttgtaaagtctacaacatcagaaatcaacccatttcctcacctcataactcttcttggtaacaagatagtttttttagatatggatcaaaatcaagttgtgtggacgttttcagcagaaagagttatcagtgccgtttgctacacagctgataatgagatgggatattatgctgaaggggactcactctttcttctgcacttgcacaatcgctctagctctgagcttttccaagattcactggtttttgatatcacagttattacaattgactggatttcaaggcacctctactttgcactgaaagaatcacaaaatggaatgcaagtatttgatgttgatcttgaacacaaggtgaaatatcccagagaggtgaagattcacaataggaattcaacaataatttctttttctgtatatcctcttttaagtcgcttgtattggacagaagtttccaattttggctaccagatgttctactacagtattatcagtcacaccttgcaccgaattctgcaacccacagctacaaaccaacaaaacaaaaggaatcaatgttcttgtaatgtgactgaatttgagttaagtggagcaatggctattgatacctctaacctagagaaaccattgatatactttgccaaagcacaagagatctgggcaatggatctggaaggctgtcagtgttggagagttatcacagtacctgctatgctcgcaggaaaaacccttgttagcttaactgtggatggagatcttatatactggatcatcacagcaaaggacagcacacagatttatcaggcaaagaaaggaaatggggccatcgtttcccaggtgaaggccctaaggagtaggcatatcttggcttacagttcagttatgcagccttttccagataaagcgtttctgtctctagcttcagacactgtggaaccaactatacttaatgccactaacactagcctcacaatcagattacctctggccaagacaaacctcacatggtatggcatcaccagccctactccaacatacctggtttattatgcagaagttaatgacaggaaaaacagctctgacttgaaatatagaattctggaatttcaggacagtatagctcttattgaagatttacaaccattttcaacatacatgatacagatagctgtaaaaaattattattcagatcctttggaacatttaccaccaggaaaagagatttggggaaaaactaaaaatggagtaccagaggcagtgcagctcattaatacaactgtgcggtcagacaccagcctcattatatcttggagagaatctcacaagccaaatggacctaaagaatcagtccgttatcagttggcaatctcacacctggccctaattcctgaaactcctctaagacaaagtgaatttccaaatggaaggctcactctccttgttactagactgtctggtggaaatatttatgtgttaaaggttcttgcctgccactctgaggaaatgtggtgtacagagagtcatcctgtcactgtggaaatgtttaacacaccagagaaaccttattccttggttccagagaacactagtttgcaatttaattggaaggctccattgaatgttaacctcatcagattttgggttgagctacagaagtggaaatacaatgagttttaccatgttaaaacttcatgcagccaaggtcctgcttatgtctgtaatatcacaaatctacaaccttatacttcatataatgtcagagtagtggtggtttataagacgggagaaaatagcacctcacttccagaaagctttaagacaaaagctggagtcccaaataaaccaggcattcccaaattactagaagggagtaaaaattcaatacagtgggagaaagctgaagataatggatgtagaattacatactatatccttgagataagaaagagcacttcaaataatttacagaaccagaatttaaggtggaagatgacatttaatggatcctgcagtagtgtttgcacatggaagtccaaaaacctgaaaggaatatttcagttcagagtagtagctgcaaataatctagggtttggtgaatatagtggaatcagtgagaatattatattagttggagatgatttttggataccagaaacaagtttcatacttactattatagttggaatatttctggttgttacaatcccactgacctttgtctggcatagaagattaaagaatcaaaaaagtgccaaggaaggggtgacagtgcttataaacgaagacaaagagttggctgagctgcgaggtctggcagccggagtaggcctggctaatgcctgctatgcaatacatactcttccaacccaagaggagattgaaaatcttcctgccttccctcgggaaaaactgactctgcgtctcttgctgggaagtggagcctttggagaagtgtatgaaggaacagcagtggacatcttaggagttggaagtggagaaatcaaagtagcagtgaagactttgaagaagggttccacagaccaggagaagattgaattcctgaaggaggcacatctgatgagcaaatttaatcatcccaacattctgaagcagcttggagtttgtctgctgaatgaaccccaatacattatcctggaactgatggagggaggagaccttcttacttatttgcgtaaagcccggatggcaacgttttatggtcctttactcaccttggttgaccttgtagacctgtgtgtagatatttcaaaaggctgtgtctacttggaacggatgcatttcattcacagggatctggcagctagaaattgccttgtttccgtgaaagactataccagtccacggatagtgaagattggagactttggactcgccagagacatctataaaaatgattactatagaaagagaggggaaggcctgctcccagttcggtggatggctccagaaagtttgatggatggaatcttcactactcaatctgatgtatggtcttttggaattctgatttgggagattttaactcttggtcatcagccttatccagctcattccaaccttgatgtgttaaactatgtgcaaacaggagggagactggagccaccaagaaattgtcctgatgatctgtggaatttaatgacccagtgctgggctcaagaacccgaccaaagacctacttttcatagaattcaggaccaacttcagttattcagaaattttttcttaaatagcatttataagtccagagatgaagcaaacaacagtggagtcataaatgaaagctttgaaggtgaagatggcgatgtgatttgtttgaattcagatgacattatgccagttgctttaatggaaacgaagaaccgagaagggttaaactatatggtacttgctacagaatgtggccaaggtgaagaaaagtctgagggtcctctaggctcccaggaatctgaatcttgtggtctgaggaaagaagagaaggaaccacatgcagacaaagatttctgccaagaaaaacaagtggcttactgcccttctggcaagcctgaaggcctgaactatgcctgtctcactcacagtggatatggagatgggtctgattaatagcgttgtttgggaaatagagagttgagataaacactctcattcagtagttactgaaagaaaactctgctagaatgataaatgtcatggtggtctataactccaaataaacaatgcaacgttcc

SEQ ID NO: 3 - DNA, 4174 bp, Homo sapiens
agtcccgcgaccgaagcagggcgcgcagcagcgctgagtgccccggaacgtgcgtcgcgcccccagtgtccgtcgcgtccgccgcgccccgggcggggatggggcggccagactgagcgccgcacccgccatccagacccgccggccctagccgcagtccctccagccgtggccccagcgcgcacgggcgatggcgaaggcgacgtccggtgccgcggggctgcgtctgctgttgctgctgctgctgccgctgctaggcaaagtggcattgggcctctacttctcgagggatgcttactgggagaagctgtatgtggaccaggcggccggcacgcccttgctgtacgtccatgccctgcgggacgcccctgaggaggtgcccagcttccgcctgggccagcatctctacggcacgtaccgcacacggctgcatgagaacaactggatctgcatccaggaggacaccggcctcctctaccttaaccggagcctggaccatagctcctgggagaagctcagtgtccgcaaccgcggctttcccctgctcaccgtctacctcaaggtcttcctgtcacccacatcccttcgtgagggcgagtgccagtggccaggctgtgcccgcgtatacttctccttcttcaacacctcctttccagcctgcagctccctcaagccccgggagctctgcttcccagagacaaggccctccttccgcattcgggagaaccgacccccaggcaccttccaccagttccgcctgctgcctgtgcagttcttgtgccccaacatcagcgtggcctacaggctcctggagggtgagggtctgcccttccgctgcgccccggacagcctggaggtgagcacgcgctgggccctggaccgcgagcagcgggagaagtacgagctggtggccgtgtgcaccgtgcacgccggcgcgcgcgaggaggtggtgatggtgcccttcccggtgaccgtgtacgacgaggacgactcggcgcccaccttccccgcgggcgtcgacaccgccagcgccgtggtggagttcaagcggaaggaggacaccgtggtggccacgctgcgtgtcttcgatgcagacgtggtacctgcatcaggggagctggtgaggcggtacacaagcacgctgctccccggggacacctgggcccagcagaccttccgggtggaacactggcccaacgagacctcggtccaggccaacggcagcttcgtgcgggcgaccgtacatgactataggctggttctcaaccggaacctctccatctcggagaaccgcaccatgcagctggcggtgctggtcaatgactcagacttccagggcccaggagcgggcgtcctcttgctccacttcaacgtgtcggtgctgccggtcagcctgcacctgcccagtacctactccctctccgtgagcaggagggctcgccgatttgcccagatcgggaaagtctgtgtggaaaactgccaggcattcagtggcatcaacgtccagtacaagctgcattcctctggtgccaactgcagcacgctaggggtggtcacctcagccgaggacacctcggggatcctgtttgtgaatgacaccaaggccctgcggcggcccaagtgtgccgaacttcactacatggtggtggccaccgaccagcagacctctaggcaggcccaggcccagctgcttgtaacagtggaggggtcatatgtggccgaggaggcgggctgccccctgtcctgtgcagtcagcaagagacggctggagtgtgaggagtgtggcggcctgggctccccaacaggcaggtgtgagtggaggcaaggagatggcaaagggatcaccaggaacttctccacctgctctcccagcaccaagacctgccccgacggccactgcgatgttgtggagacccaagacatcaacatttgccctcaggactgcctccggggcagcattgttgggggacacgagcctggggagccccgggggattaaagctggctatggcacctgcaactgcttccctgaggaggagaagtgcttctgcgagcccgaagacatccaggatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccggtgcccgccggccctcgctggactccatggagaaccaggtctccgtggatgccttcaagatcctggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagcttcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctagcaccgctgtcccctctgcactatccttcctctctgtgatgctttttaaaaatgtttctggtctgaacaaaaccaaagtctgctctgaacctttttatttgtaaatgtctgactttgcatccagtttacatttaggcattattgcaactatgtttttctaaaaggaagtgaaaataagtgtaattaccacattgcccagcaacttaggatggtagaggaaaaaacagatcagggcggaactctcaggggagaccaagaacaggttgaataaggcgcttctggggtgggaatcaagtcatagtacttctactttaactaagtggataaatatacaaatctggggaggtattcagttgagaaaggagccaccagcaccactcagcctgcactgggagcacagccaggttcccccagacccctcctgggcaggcaggtgcctctcagaggccacccggcactggcgagcagccactggccaagcctcagccccagtcccagccacatgtcctccatcaggggtagcgaggttgcaggagctggctggccctgggaggacgcacccccactgctgttttcacatcctttcccttacccaccttcaggacggttgtcacttatgaagtcagtgctaaagctggagcagttgctttttgaaagaacatggtctgtggtgctgtggtcttacaatggacagtaaatatggttcttgccaaaactccttcttttgtctttgattaaatactagaaatttaaaaaaaaaaaaaaa

SEQ ID NO: 4 - DNA, 5629 bp, Homo sapiens
agtcccgcgaccgaagcagggcgcgcagcagcgctgagtgccccggaacgtgcgtcgcgcccccagtgtccgtcgcgtccgccgcgccccgggcggggatggggcggccagactgagcgccgcacccgccatccagacccgccggccctagccgcagtccctccagccgtggccccagcgcgcacgggcgatggcgaaggcgacgtccggtgccgcggggctgcgtctgctgttgctgctgctgctgccgctgctaggcaaagtggcattgggcctctacttctcgagggatgcttactgggagaagctgtatgtggaccaggcggccggcacgcccttgctgtacgtccatgccctgcgggacgcccctgaggaggtgcccagcttccgcctgggccagcatctctacggcacgtaccgcacacggctgcatgagaacaactggatctgcatccaggaggacaccggcctcctctaccttaaccggagcctggaccatagctcctgggagaagctcagtgtccgcaaccgcggctttcccctgctcaccgtctacctcaaggtcttcctgtcacccacatcccttcgtgagggcgagtgccagtggccaggctgtgcccgcgtatacttctccttcttcaacacctcctttccagcctgcagctccctcaagccccgggagctctgcttcccagagacaaggccctccttccgcattcgggagaaccgacccccaggcaccttccaccagttccgcctgctgcctgtgcagttcttgtgccccaacatcagcgtggcctacaggctcctggagggtgagggtctgcccttccgctgcgccccggacagcctggaggtgagcacgcgctgggccctggaccgcgagcagcgggagaagtacgagctggtggccgtgtgcaccgtgcacgccggcgcgcgcgaggaggtggtgatggtgcccttcccggtgaccgtgtacgacgaggacgactcggcgcccaccttccccgcgggcgtcgacaccgccagcgccgtggtggagttcaagcggaaggaggacaccgtggtggccacgctgcgtgtcttcgatgcagacgtggtacctgcatcaggggagctggtgaggcggtacacaagcacgctgctccccggggacacctgggcccagcagaccttccgggtggaacactggcccaacgagacctcggtccaggccaacggcagcttcgtgcgggcgaccgtacatgactataggctggttctcaaccggaacctctccatctcggagaaccgcaccatgcagctggcggtgctggtcaatgactcagacttccagggcccaggagcgggcgtcctcttgctccacttcaacgtgtcggtgctgccggtcagcctgcacctgcccagtacctactccctctccgtgagcaggagggctcgccgatttgcccagatcgggaaagtctgtgtggaaaactgccaggcattcagtggcatcaacgtccagtacaagctgcattcctctggtgccaactgcagcacgctaggggtggtcacctcagccgaggacacctcggggatcctgtttgtgaatgacaccaaggccctgcggcggcccaagtgtgccgaacttcactacatggtggtggccaccgaccagcagacctctaggcaggcccaggcccagctgcttgtaacagtggaggggtcatatgtggccgaggaggcgggctgccccctgtcctgtgcagtcagcaagagacggctggagtgtgaggagtgtggcggcctgggctccccaacaggcaggtgtgagtggaggcaaggagatggcaaagggatcaccaggaacttctccacctgctctcccagcaccaagacctgccccgacggccactgcgatgttgtggagacccaagacatcaacatttgccctcaggactgcctccggggcagcattgttgggggacacgagcctggggagccccgggggattaaagctggctatggcacctgcaactgcttccctgaggaggagaagtgcttctgcgagcccgaagacatccaggatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccggtgcccgccggccctcgctggactccatggagaaccaggtctccgtggatgccttcaagatcctggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagcttcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggcatgtcagacccgaactggcctggagagagtcctgtaccactcacgagagctgatggcactaacactgggtttccaagatatccaaatgatagtgtatatgctaactggatgctttcaccctcagcggcaaaattaatggacacgtttgatagttaacatttctttgtgaaaggtaatggactcacaaggggaagaaacatgctgagaatggaaagtctaccggccctttctttgtgaacgtcacattggccgagccgtgttcagttcccaggtggcagactcgtttttggtagtttgttttaacttccaaggtggttttacttctgatagccggtgattttccctcctagcagacatgccacaccgggtaagagctctgagtcttagtggttaagcattcctttctcttcagtgcccagcagcacccagtgttggtctgtgtccatcagtgaccaccaacattctgtgttcacatgtgtgggtccaacacttactacctggtgtatgaaattggacctgaactgttggatttttctagttgccgccaaacaaggcaaaaaaatttaaacatgaagcacacacacaaaaaaggcagtaggaaaaatgctggccctgatgacctgtccttattcagaatgagagactgcggggggggcctgggggtagtgtcaatgcccctccagggctggaggggaagaggggccccgaggatgggcctgggctcagcattcgagatcttgagaatgatttttttttaatcatgcaacctttccttaggaagacatttggttttcatcatgattaagatgattcctagatttagcacaatggagagattccatgccatctttactatgtggatggtggtatcagggaagagggctcacaagacacatttgtcccccgggcccaccacatcatcctcacgtgttcggtactgagcagccactacccctgatgagaacagtatgaagaaagggggctgttggagtcccagaattgctgacagcagaggctttgctgctgtgaatcccacctgccaccagcctgcagcacaccccacagccaagtagaggcgaaagcagtggctcatcctacctgttaggagcaggtagggcttgtactcactttaatttgaatcttatcaacttactcataaagggacaggctagctagctgtgttagaagtagcaatgacaatgaccaaggactgctacacctctgattacaattctgatgtgaaaaagatggtgtttggctcttatagagcctgtgtgaaaggcccatggatcagctcttcctgtgtttgtaatttaatgctgctacaagatgtttctgtttcttagattctgaccatgactcataagcttcttgtcattcttcattgcttgtttgtggtcacagatgcacaacactcctccagtcttgtgggggcagcttttgggaagtctcagcagctcttctggctgtgttgtcagcactgtaacttcgcagaaaagagtcggattaccaaaacactgcctgctcttcagacttaaagcactgataggacttaaaatagtctcattcaaatactgtattttatataggcatttcacaaaaacagcaaaattgtggcattttgtgaggccaaggcttggatgcgtgtgtaatagagccttgtggtgtgtgcgcacacacccagagggagagtttgaaaaatgcttattggacacgtaacctggctctaatttgggctgtttttcagatacactgtgataagttcttttacaaatatctatagacatggtaaacttttggttttcagatatgcttaatgatagtcttactaaatgcagaaataagaataaactttctcaaattattaaaaatgcctacacagtaagtgtgaattgctgcaacaggtttgttctcaggagggtaagaactccaggtctaaacagctgacccagtgatggggaatttatccttgaccaatttatccttgaccaataacctaattgtctattcctgagttataaaagtccccatccttattagctctactggaattttcatacacgtaaatgcagaagttactaagtattaagtattactgagtattaagtagtaatctgtcagttattaaaatttgtaaaatctatttatgaaaggtcattaaaccagatcatgttcctttttttgtaatcaaggtgactaagaaaatcagttgtgtaaataaaatcatgtatcataaaaaaaaaaaaaaaa

SEQ ID NO: 5 - DNA, 110 bp, Unknown
  Description of Unknown PIK3CA polynucleotide
ttcgaaagaccctagccttagataaaactgagcaagaggctttggagtatttcatgaaacaaatgaatgatgcacatcatggtggctggacaacaaaaatggattggatc

SEQ ID NO: 6 - DNA, 110 bp, Unknown
  Description of Unknown PIK3CA mutant polynucleotide
ttcgaaagaccctagccttagataaaactgagcaagaggctttggagtatttcatgaaacaaattaatgatgcacatcatggtggctggacaacaaaaatggattggatc

SEQ ID NO: 7 - DNA, 110 bp, Unknown
  Description of Unknown PIK3CA mutant polynucleotide
ttcgaaagaccctagccttagataaaactgagcaagagactttggagtatttcatgaaacaaattaatgatgcacatcatggtggctggacaacaaaaatggattggatc

SEQ ID NO: 8 - DNA, 110 bp, Unknown
  Description of Unknown PIK3CA mutant polynucleotide
ttcgaaagaccctagccttagataaaactgaacaagaggctttggagtatttcatgaaacaaattaatgatgcacatcatggtggctggacaacaaaaatggattggatc

SEQ ID NO: 9 - DNA, 110 bp, Unknown
  Description of Unknown PIK3CA mutant polynucleotide
ttcgaaagacactagccgtagataaaactgagcaagaggctttggagtatttcatgaaacaaattaatgatgcacatcatggtggctggacaacaaaaatggattggatc

SEQ ID NO: 10 - DNA, 110 bp, Unknown
  Description of Unknown PIK3CA mutant polynucleotide
ttcgaaagaccctagccttagataaaactgagcaagaggctttggagtatttcatgaaacaaattaatgatgcacatcatagtggctggacaacaaaaatggattggatc

SEQ ID NO: 11 - DNA, 109 bp, Unknown
  Description of Unknown PIK3CA mutant polynucleotide
ttcgaaagaccctagccgtagataaactgagcaagaggctttggagtatttcatgaaacaaattaatgatgcacatcatggtggctggacaacaaaaatggattggatc

SEQ ID NO: 12 - protein, 35 aa, Unknown
  Description of Unknown PIK3CA polypeptide
RKTLALDKTEQEALEYEMKQMNDAHHGGWTTKMDW

(19) United States
(12) Patent Application Publication
(10) Pub. No.: US 2015/0211061 A1
(43) Pub. Date: Jul. 30, 2015

(54) METHODS FOR DETERMINING A NUCLEOTIDE SEQUENCE

(71) Applicant: The General Hospital Corporation, Boston, MA (US)

(72) Inventors: Anthony John Iafrate, Newton, MA (US); Long Phi Le, Boston, MA (US); Zongli Zheng, Boston, MA (US)

(21) Appl. No.: 14/605,482

(22) Filed: Jan. 26, 2015

Related U.S. Application Data
(60) Provisional application No. 61/931,943, filed on Jan. 27, 2014.

(51) Int. Cl. C12Q 1/68

(52) U.S. Cl. CPC C12Q 1/6874

(57) ABSTRACT

Aspects of the technology disclosed herein relate to methods for preparing and analyzing nucleic acids. In some embodiments, methods for preparing nucleic acids for sequence analysis (e.g., using next-generation sequencing) are provided herein.

### CROSS-REFERENCE TO RELATED APPLICATIONS

This application claims benefit under 35 U.S.C. §119(e) of U.S. Provisional Application No. 61/931,943 filed Jan. 27, 2014, the contents of which are incorporated herein by reference in their entirety.

### TECHNICAL FIELD

The technology described herein relates to methods of determining oligonucleotide sequences and/or preparing and analyzing nucleic acids.

### BACKGROUND

Target enrichment prior to next-generation sequencing is more cost-effective than whole genome, whole exome, and whole transcriptome sequencing and therefore more practical for broad implementation; both for research discovery and clinical applications. For example, high coverage depth afforded by target enrichment approaches enables a wider dynamic range for allele counting (in gene expression and copy number assessment) and detection of low frequency mutations, a critical feature for evaluating somatic mutations in cancer. Examples of current enrichment protocols for next generation sequencing include hybridization-based capture assays (TruSeq Capture, Illumina; SureSelect Hybrid Capture, Agilent) and polymerase chain reaction (PCR)-based assays (HaloPlex, Agilent; AmpliSeq, Ion Torrent; TruSeq Amplicon, Illumina; emulsion/digital PCR, Raindance). Hybridization-based approaches capture not only the targeted sequences covered by the capture probes but also near off-target bases that consume sequencing capacity. In addition, these methods are relatively time-consuming, labor-intensive, and suffer from a relatively low level of specificity. A PCR amplification based approach is simpler and faster but by conventional design requires the use of both forward and reverse primers flanking the target loci. In particular, for detection of genomic rearrangements with unknown fusion partners, PCR is not applicable.

### SUMMARY

The technology described herein is directed to methods of determining oligonucleotide sequences. In some embodiments, the methods described herein relate to enriching target sequences prior to sequencing the oligonucleotide sequences.

Aspects of the technology disclosed herein relate to methods for preparing and analyzing nucleic acids. In some embodiments, methods for preparing nucleic acids for sequence analysis (e.g., using next-generating sequencing) are provided herein. In some embodiments, technology described herein is directed to methods of determining nucleotide sequences of nucleic acids. In some embodiments, the methods described herein relate to enriching target nucleic acids prior to sequencing.

In one aspect, described herein is a method of determining the nucleotide sequence contiguous to a known target nucleotide sequence, the method comprising; (a) hybridizing a target nucleic acid molecule comprising the known target nucleotide sequence with a population of tailed random primers; (b) extension of a hybridized tailed random primer using the portion of the target nucleic acid molecule downstream of the site of hybridization as a template; (c) amplifying a portion of the target nucleic acid molecule and the tailed random primer sequence with a first tail primer and a first target-specific primer; (d) amplifying a portion of the amplicon resulting from step (c) with a second tail primer and a second target-specific primer; (c) sequencing the amplified portion from step (d) using a first and second sequencing primer; wherein the population of tailed random primers comprises single-stranded oligonucleotide molecules having a 5′ nucleic acid sequence identical or complementary to a first sequencing primer and a 3′ nucleic acid sequence comprising from about 6 to about 12 random nucleotides; wherein the first target-specific primer comprises a nucleic acid sequence that can specifically anneal to the known target nucleotide sequence of the target nucleic acid at the annealing temperature; wherein the second target-specific primer comprises a 3′ portion comprising a nucleic acid sequence that can specifically anneal to a portion of the known target nucleotide sequence comprised by the amplicon resulting from step (c), and a 5′ portion comprising a nucleic acid sequence that is identical to a second sequencing primer and the second target-specific primer is nested with respect to the first target-specific primer; wherein the first tail primer comprises a nucleic acid sequence identical or complementary to all or a portion of the 5′ portion of the tailed random primer; and wherein the second tail primer comprises a nucleic acid sequence identical or complementary to a portion of the first sequencing primer and is nested with respect to the first tail primer.

In some embodiments, the 5′ nucleic acid sequence of the tailed random primers is identical to a first sequencing primer. In some embodiments, the first tail primer comprises a nucleic acid sequence identical to the 5′ portion of the tailed random primer. In some embodiments, the second tail primer comprises a nucleic acid sequence identical to a portion of the first sequencing primer. In some embodiments, the each tailed random primer further comprises a spacer nucleic acid sequence between the 5′ nucleic acid sequence identical or complementary to a first sequencing primer and the 3′ nucleic acid sequence comprising about 6 to about 12 random nucleotides. In some embodiments, the unhybridized primers are removed from the reaction after an extension step. In some embodiments, the second tail primer is nested with respect to the first tail primer by at least 3 nucleotides. In some embodiments, the first target-specific primer further comprises a 5′ tag sequence portion comprising a nucleic acid sequence of high GC content which is not substantially complementary to or substantially identical to any other portion of any of the primers. In some embodiments, the second tail primer is identical to the full-length first sequencing primer. In some embodiments, the portions of the target-specific primers that specifically anneal to the known target will anneal specifically at a temperature of about 65° C. in a PCR buffer. In some embodiments, the sample comprises genomic DNA. In some embodiments, the sample comprises RNA and the method further comprises a first step of subjecting the sample to a reverse transcriptase regimen. In some embodiments, the nucleic acids present in the sample have not been subjected to shearing or digestion. In some embodiments, the sample comprises single-stranded gDNA or cDNA. In some embodiments, the reverse transcriptase regimen comprises the use of random hexamers. In some embodiments, a gene rearrangement comprises the known target sequence. In some embodiments, the gene rearrangement is present in a nucleic acid selected from the group consisting of: genomic DNA; RNA; and cDNA. In some embodiments, the gene rearrangement comprises an oncogene. In some embodiments, the gene rearrangement comprises a fusion oncogene. In some embodiments, the nucleic acid product is sequenced by a next-generation sequencing method. In some embodiments, the next-generation sequencing method comprises a method selected from the group consisting of: Ion Torrent, Illumina, SOLiD, 454; Massively Parallel Signature Sequencing solid-phase, reversible dye-terminator sequencing; and DNA nanoball sequencing. In some embodiments, the first and second sequencing primers are compatible with the selected next-generation sequencing method. In some embodiments, the method comprises contacting the sample, or separate portions of the sample, with a plurality of sets of first and second target-specific primers. In some embodiments, the method comprises contacting a single reaction mixture comprising the sample with a plurality of sets of first and second target-specific primers. In some embodiments, the plurality of sets of first and second target-specific primers specifically anneal to known target nucleotide sequences comprised by separate genes. In some embodiments, at least two sets of first and second target-specific primers specifically anneal to different portions of a known target nucleotide sequence. In some embodiments, at least two sets of first and second target-specific primers specifically anneal to different portions of a single gene comprising a known target nucleotide sequence. In some embodiments, at least two sets of first and second target-specific primers specifically anneal to different exons of a gene comprising a known nucleotide target sequence. In some embodiments, the plurality of first target-specific primers comprise identical 5′ tag sequence portions. In some embodiments, each amplification step comprises a set of cycles of a PCR amplification regimen from 5 cycles to 20 cycles in length. In some embodiments, the target-specific primers and the tail primers are designed such that they will specifically anneal to their complementary sequences at an annealing temperature of from about 61 to 72° C. In some embodiments, the target-specific primers and the tail primers are designed such that they will specifically anneal to their complementary sequences at an annealing temperature of about 65° C.

In some embodiments, the target nucleic acid molecule is from a sample, optionally which is a biological sample obtained from a subject. In some embodiments, the sample is obtained from a subject in need of treatment for a disease associated with a genetic alteration. In some embodiments, the disease is cancer. In some embodiments, the sample comprises a population of tumor cells. In some embodiments, the sample is a tumor biopsy. In some embodiments, the cancer is lung cancer. In some embodiments, a disease-associated gene comprises the known target sequence. In some embodiments, the target nucleic acid is a ribonucleic acid. In some embodiments, the target nucleic acid is a deoxyribonucleic acid. In some embodiments, the target nucleic acid is a messenger RNA encoded from a chromosomal segment that comprises a genetic rearrangement. In some embodiments, the target nucleic acid is a chromosomal segment that comprises a portion of a genetic rearrangement.

In one aspect, described herein is a method of preparing nucleic acids for analysis, the method comprising: contacting a nucleic acid template comprising with a plurality of different primers that share a common sequence that is 5′ to different hybridization sequences, under conditions to promote template-specific hybridization and extension of at least one of the plurality of different primers; contacting the extension product of the first step with a first tail primer and a first target-specific primer under conditions to promote template-specific hybridization and extension from the first tail primer and first target-specific primer; contacting the extension product of the second step with a second tail primer and a second target-specific primer under conditions to promote template-specific hybridization and extension from the second tail primer and second target-specific primer; wherein the first target-specific primer comprises a nucleic acid sequence that can specifically anneal to a known target nucleotide sequence of the target nucleic acid at the annealing temperature; wherein the second target-specific primer comprises a 3′ portion comprising a nucleic acid sequence that can specifically anneal to a portion of the known target nucleotide sequence comprised by the amplicon resulting from the second step, and a 5′ portion comprising a nucleic acid sequence that is identical to a second sequencing primer and the second target-specific primer is nested with respect to the first target-specific primer; wherein the first tail primer comprises a nucleic acid sequence identical or complementary to the common sequence of the primers of the first step; and wherein the second tail primer comprises a nucleic acid sequence identical or complementary to a portion of the first sequencing primer and is nested with respect to the first tail primer. In some embodiments, the target nucleic acid is a ribonucleic acid. In some embodiments, the target nucleic acid is a deoxyribonucleic acid. In some embodiments, the target nucleic acid is a messenger RNA encoded from a chromosomal segment that comprises a genetic rearrangement. In some embodiments, the target nucleic acid is a chromosomal segment that comprises a portion of a genetic rearrangement. In some embodiments, the genetic rearrangement is an inversion, deletion, or translocation. In some embodiments, the method further comprises amplifying one or more of the extension products In some embodiments, each of the primers of the first step further comprises a spacer nucleic acid sequence between the common sequence and the hybridization sequence, the spacer sequence comprising about 6 to about 12 random nucleotides. In some embodiments, the unhybridized primers are removed from the reaction after extension. In some embodiments, the second tail primer is nested with respect to the first tail primer by at least 3 nucleotides. In some embodiments, the first target-specific primer further comprises a 5′ tag sequence portion comprising a nucleic acid sequence of high GC content which is not substantially complementary to or substantially identical to any other portion of any of the primers. In some embodiments, the portions of the target-specific primers that specifically anneal to the known target will anneal specifically at a temperature of about 65° C. in a PCR buffer.

### BRIEF DESCRIPTION OF THE DRAWINGS

FIG. 1 depicts a schematic of an exemplary method of amplifying and sequencing a target oligonucleotide as described herein.

FIG. 2 depicts sequencing data obtained in accordance with the methods described herein. Random errors in amplification or sequencing can be readily distinguished from actual mutations.

FIG. 3 depicts a schematic of an exemplary, non-limiting method of amplifying a target oligonucleotide sequence as described herein.

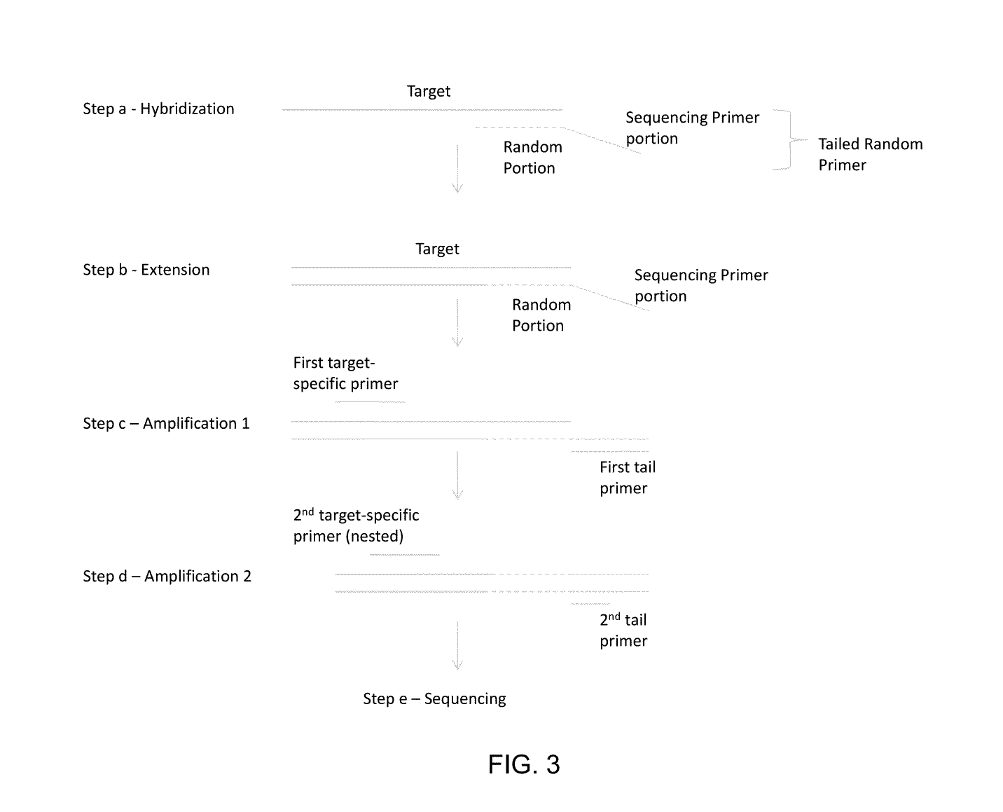

FIG. 4 depicts a non-limiting embodiment of a work flow for amplifying and sequencing target nucleic acids that are flanked by an unknown fusion partner (e.g. a 5′ unknown fusion partner), as described herein.

### DETAILED DESCRIPTION

Embodiments of the technology described herein relate to methods of determining (i.e. sequencing) oligonucleotide sequences. In some embodiments, the methods described herein relate to methods of enriching target sequences prior to a sequencing step. In some embodiments, the sequence of one end of the target sequence to be enriched is not known prior to the sequencing step. Aspects of the technology disclosed herein relate to methods for preparing and analyzing nucleic acids. In some embodiments, methods Provided herein are useful for determining unknown nucleotide sequences contiguous to (adjacent to) a known target nucleotide sequence. Traditional sequencing methods generate sequence information randomly (e.g. “shotgun” sequencing) or between two known sequences which are used to design primers. In contrast, methods described herein, in some embodiments, allow for determining the nucleotide sequence (e.g. sequencing) upstream or downstream of a single region of known sequence with a high level of specificity and sensitivity. Accordingly, in some embodiments, methods provided herein are useful for determining the sequence of fusions (e.g., fusion mRNAs) that result from gene arrangements (e.g., rearrangements that give rise to cancer or other disorders).

In some embodiments, the methods described herein relate to a method of enriching specific nucleotide sequences prior to determining the nucleotide sequence using a next-generation sequencing technology. In some embodiments, the methods of enriching specific nucleotide sequences do not comprise hybridization enrichment.

In some embodiments, the technology described herein can relate to a method of determining the nucleotide sequence contiguous to a known target nucleotide sequence, the method comprising; (a) hybridizing a target nucleic acid molecule comprising the known target nucleotide sequence with a population of tailed random primers; (b) extension of a hybridized tailed random primer using the portion of the target nucleic acid molecule downstream of the site of hybridization as a template, thereby producing a primary extension product. In some embodiments, the methods further comprise amplifying a portion of the target nucleic acid molecule comprised by the primary extension product and the tailed random primer sequence with a first tail primer and a first target-specific primer, thereby producing a first amplicon. In some embodiments, the methods further comprise amplifying a portion of the first amplicon with a second tail primer and a second target-specific primer, thereby producing a second amplicon.

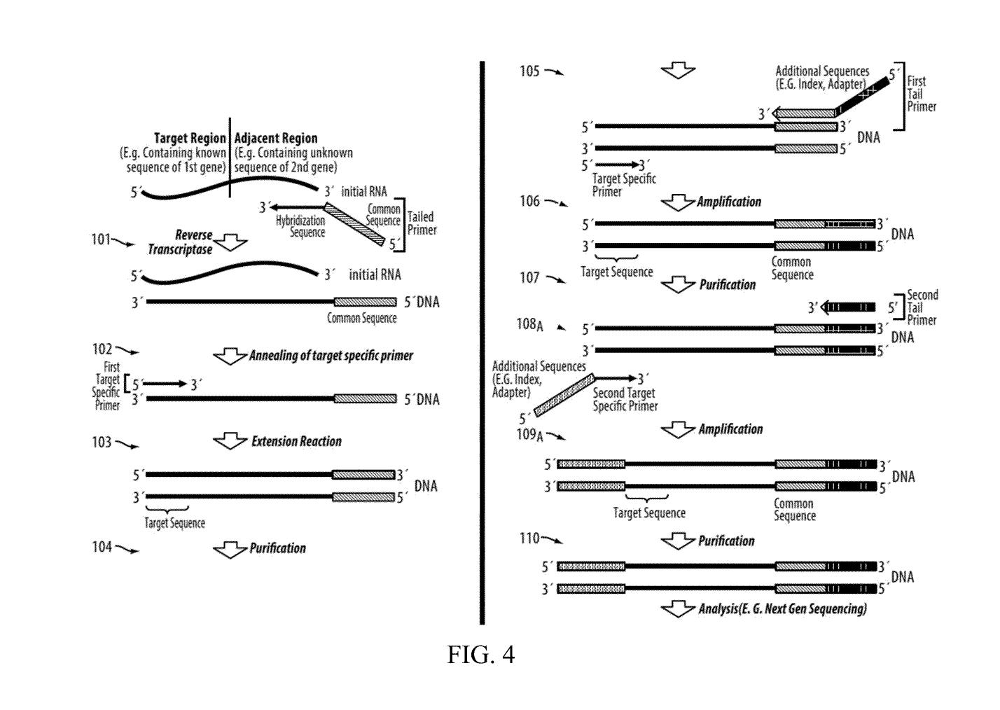

In some embodiments, methods are provided for preparing nucleic acids that have a target region 5′ to an adjacent region (e.g., an adjacent region of unknown sequence). For example, FIG. 4 presents schematics of exemplary methods of amplifying target nucleic acids that have a known target region 5′ to an adjacent region (e.g., for purposes of sequencing the adjacent region). At step 101, initial RNA is obtained or provided in a sample and is used as a template. RNA template is exposed to a plurality of tailed primers (e.g., tailed random primers) that comprise a common sequence that is 5′ to different hybridization sequences and shared between all of the tailed primers of the population. In some embodiments, at least one primer hybridizes to an RNA molecule and primes a reverse transcriptase reaction to produce a complementary DNA strand.

In step 102, DNA molecules produced by reverse transcription are contacted by one or more initial target-specific primers which may or may not be the same as the first target-specific primer. In step 103, hybridization of the initial target-specific primer to a portion of the target nucleic acid primes an extension reaction using a DNA molecule as a template to produce a complementary DNA strand. Extension products are purified in step 104.

In step 105, DNA molecules are contacted by a first target-specific primer and a first tail primer. The first target-specific primer hybridizes to a portion of the target nucleic acid. In some embodiments, pools of different first target-specific primers can be used that hybridize to different portions of a target nucleic acid. In some embodiments, use of different target specific primers can be advantageous because it allows for generation of different extension products having overlapping but staggered sequences relative to a target nucleic acid. In some embodiments, different extension products can be sequenced to produce overlapping sequence reads. In some embodiments, overlapping sequence reads can be evaluated to assess accuracy of sequence information, fidelity of nucleic acid amplification, and/or to increase confidence in detecting mutations, such as detecting locations of chromosomal rearrangements (e.g., fusion breakpoints). In some embodiments, pools of different first target-specific primers can be used that hybridize to different portions of different target nucleic acids present in sample. In some embodiments, use of pools of different target-specific primers is advantageous because it facilitates processing (e.g., amplification) and analysis of different target nucleic acids in parallel. In some embodiments, up to 2, up to 3, up to 4, up to 5, up to 6, up to 7, up to 8, up to 9, up to 10, up to 15, up to 20, up to 100 or more pools of different first target-specific primers are used. In some embodiments, 2 to 5, 2 to 10, 5 to 10, 5 to 15, 10 to 15, 10 to 20, 10 to 100, 50 to 100, or more pools of different first target-specific primers are used.

In FIG. 4, a first tail primer hybridizes to at least a portion of a DNA molecule provided by the tail portion of the tailed primer of step 101. In some embodiments, the first tail primer hybridizes to the common sequence provided by the tail of the one or more primers of step 101. In some embodiments, a nested target specific primer (nested with respect to the target specific primer of step 102) is used in step 105. In some embodiments, a first tail primer may comprise an additional sequence 5′ to the hybridization sequence that may include index, adapter sequences, or sequencing primer sites, for example. In step 106, hybridization of the first target-specific primer and the first tail nucleic acid molecule in a polymerase chain reaction (PCR). In some embodiments, amplified products are purified in step 109.

In FIG. 4 at step 108_{A}, amplified DNA products of step 106 (e.g., as purified in step 107) are contacted with a second target-specific primer and a second tail primer. In some embodiments, the second target-specific primer hybridizes to a sequence that is present within the template DNA molecule 3′ of the sequence of the first target-specific primer such that the reactions are nested. In step 109_{A}, the amplified DNA products of step 106 (e.g., as purified in step 107) are amplified by PCR in which the extensions are primed by the second target-specific primer and a second tail primer. In some embodiments, a portion of the amplified product from step 106 is further amplified. In some embodiments, a third primer is used that hybridizes to the common tail in the second target specific primer and adds additional sequences such as adapters, etc.

In some embodiments, the second target-specific primer comprises a nucleotide sequence 5′ to the target-specific sequence that comprises an index or adapter sequence. In some embodiments, the second tail primer hybridizes to a sequence that is present within the template DNA molecule 3′ of the sequence of the first tail primer such that the reactions are nested. In such embodiments, a portion of the product from step 105 is amplified. In some embodiments, the second tail primer may comprise additional sequences 5′ to the hybridization sequence that may include index, adapter sequences or sequencing primer sites. Hybridization of the second target-specific primer and the second tail primer allows for exponential amplification of a portion of the target nucleic acid molecule in a PCR reaction. The products are purified in reaction 110 and ready for analysis. For example, productions purified in step 110 can be sequenced (e.g., using a next generation sequencing platform.)

In some embodiments, steps 101-103, 105-106, and 108-109 are performed consecutively in a single reaction tube without any intervening purification steps. In some embodiments, all of the components involved in steps 101-103, 105-106, and 108-109 are present at the outset and throughout the reaction. In some embodiments, steps 101-103 are performed consecutively in a single reaction tube. In some embodiments, all of the components involved in steps 101-103 are present at the outset and throughout the reaction. In some embodiments, steps 105-106 are performed consecutively in a single reaction tube. In some embodiments, all of the components involved in steps 105-106 are present at the outset and throughout the reaction. In some embodiments, steps 108-109 are performed consecutively in a single reaction tube. In some embodiments, all of the components involved in steps 108-109 are present at the outset and throughout the reaction.

In some embodiments, methods are provided herein that involve determining the nucleotide sequence contiguous to (adjacent to) a known target nucleotide sequence.

In some embodiments, one or more target-specific primers used in the methods may be nested with respect to one or more other target-specific primers. For example, in some embodiments, a second target-specific primer is internal to a first target-specific primer. In some embodiments, target-specific primers are the same. In some embodiments, target-specific primers are nested but overlapping with respect to target complementarity. In some embodiments, target-specific primers are nested and non-overlapping. In some embodiments, combinations of identical and nested target specific primers are used in the same or different amplification steps. In some embodiments, nesting of primers increases target specificity. In some embodiments, the methods further comprise sequencing the second amplicon (e.g. the amplified portion from step (d)) using a first and second sequencing primer.

In some embodiments, the population of tailed random primers comprises single-stranded oligonucleotide molecules having a 5′ nucleotide sequence identical to a first sequencing primer and a 3′ nucleotide comprising from random nucleotides (e.g., about 6 to about 12 random nucleotides). In some embodiments, the first target-specific primer comprises a nucleic acid sequence that can specifically anneal to the known nucleotide sequence of the target nucleic acid at an appropriate annealing temperature. In some embodiments, the second target-specific primer comprises a 3′ portion comprising a nucleic acid sequence that can specifically anneal to a portion of the known target nucleotide sequence comprised by the first amplicon (e.g., the amplicon resulting from step (c)), and a 5′ portion comprising a nucleic acid sequence that is identical to a second sequencing primer and the second target-specific primer is nested with respect to the first target-specific primer. In some embodiments, the first tail primer comprises a nucleic acid sequence identical or complementary to all or a portion of the 5′ portion of the tailed random primer, e.g. the first tail primer comprises a nucleic acid sequence identical to the 5′ portion of the tailed random primer or the first tail primer comprises a nucleic acid sequence which is nested with respect to the 5′ portion of the tailed random primer. In some embodiments, the first tail primer comprises a nucleic acid sequence identical to the common sequence of the tail of the tailed random primer. In some embodiments, the first tail primer consists essentially of a nucleic acid sequence identical to the common sequence of the tail of the tailed random primer. In some embodiments, the first tail primer consists of a nucleic acid sequence identical to the common sequence of the tail of the tailed random primer. In some embodiments, the common sequence on the tailed random primer is the exact match of the common sequence on the first tail primer. In some embodiments, the second tail primer comprises a nucleic acid sequence identical to a portion of the first sequencing primer. In some embodiments, the second tail primer comprises a nucleic acid sequence identical to the first sequencing primer. In some embodiments, the second tail primer is nested with respect to the first tail primer. In some embodiments, the second tail primer comprises a nucleic acid sequence identical to a portion of the first sequencing primer and is nested with respect to the first tail primer.

As used herein, the term “target nucleic acid” refers to a nucleic acid molecule of interest (e.g., an nucleic acid to be analyzed). In some embodiments, a target nucleic acid comprises both a target nucleotide sequence (e.g., a known or predetermined nucleotide sequence or known target nucleotide sequence) and an adjacent nucleotide sequence which is to be determined (which may be referred to as an unknown sequence). A target nucleic acid can be of any appropriate length. In some embodiments, a target nucleic acid is double-stranded. In some embodiments, the target nucleic acid is DNA. In some embodiments, the target nucleic acid is genomic or chromosomal DNA (gDNA). In some embodiments, the target nucleic acid can be complementary DNA (cDNA). In some embodiments, the target nucleic acid is single-stranded. In some embodiments, the target nucleic acid can be RNA, e.g., mRNA, rRNA, tRNA, long non-coding RNA, microRNA.

As used herein, the term “known target nucleotide sequence” refers to a portion of a target nucleic acid for which the sequence (e.g. the identity and order of the nucleotide bases of the nucleic acid) is known. For example, in some embodiments, a known target nucleotide sequence is a nucleotide sequence of a nucleic acid that is known or that has been determined in advance of an interrogation of an adjacent unknown sequence of the nucleic acid. A known target nucleotide sequence can be of any appropriate length.

In some embodiments, a target nucleotide sequence (e.g., a known target nucleotide sequence) has a length of 10 or more nucleotides, 30 or more nucleotides, 40 or more nucleotides, 50 or more nucleotides, 100 or more nucleotides, 200 or more nucleotides, 300 or more nucleotides, 400 or more nucleotides, 500 or more nucleotides. In some embodiments, a target nucleotide sequence (e.g., a known target nucleotide sequence) has a length in range of 10 to 100 nucleotides, 10 to 500 nucleotides, 10 to 1000 nucleotides, 100 to 500 nucleotides, 100 to 1000 nucleotides, 500 to 1000 nucleotides, 500 to 5000 nucleotides.

In some embodiments, methods are provided herein for determining sequences of contiguous (or adjacent) portions of a nucleic acid. As used herein, the term “nucleotide sequence contiguous to” refers to a nucleotide sequence of a nucleic acid molecule (e.g., a target nucleic acid) that is immediately upstream or downstream of another nucleotide sequence (e.g., a known nucleotide sequence). In some embodiments, a nucleotide sequence contiguous to a known target nucleotide sequence may be of any appropriate length. In some embodiments, a nucleotide sequence contiguous to a known target nucleotide sequence comprises 1 kb or less of nucleotide sequence, e.g. 1 kb or less of nucleotide sequence, 750 bp or less of nucleotide sequence, 500 bp or less of nucleotide sequence, 400 bp or less of nucleotide sequence, 300 bp or less of nucleotide sequence, 200 bp or less of nucleotide sequence, 100 bp or less of nucleotide sequence. In some embodiments, in which a sample comprises different target nucleic acids comprising a known target nucleotide sequence (e.g. a cell in which a known target nucleotide sequence occurs multiple times in its genome, or on separate, non-identical chromosomes), there may be multiple sequences which comprise “a nucleotide sequence contiguous to” the known target nucleotide sequence. As used herein, the term “determining a (or the) nucleotide sequence,” refers to determining the identity and relative positions of the nucleotide bases of a nucleic acid.

In some embodiments of methods disclosed herein one or more tailed random primers are hybridized to a nucleic acid template (e.g., a template comprising a strand of a target nucleic acid (e.g., step (a)). In some embodiments, a target nucleic acid is present in or obtained from a sample comprising a plurality of nucleic acids, one or more of which plurality do not comprise the target nucleic acid. In some embodiments, one or more primers (e.g., one or more tailed random primers) hybridize to substantially all of the nucleic acids in a sample. In some embodiments, one or more primers (e.g., one or more tailed random primers) hybridize to nucleic acids that comprise a target nucleic acid and to nucleic acids that do not comprise the target nucleotide sequence.

Aspects of certain methods disclosed herein relate to contacting a nucleic acid template with a plurality of different primers that share a common sequence that is 5′ (or upstream) to different hybridization sequences. In some embodiments the plurality of different primers may be referred to as a population of different primers. In some embodiments, the common sequence may be referred to as a tail, as such the primers are referred to as “tailed primers.” In some embodiments, different hybridization sequences of a population comprise nucleotide sequences that occur randomly or pseudorandomly within the population. In some embodiments, nucleotide sequences that occur randomly within a population contain no recognizable regularities, such that, for each nucleotide of each sequence in the population, there is an equal likelihood that the nucleotide comprises a base that is complementary with A, T, G, or C. In such embodiments, it should be appreciated that each nucleotide comprising a base that is complementary with A, T, G, or C may be a naturally occurring nucleotide, a non-naturally occurring nucleotide or a modified nucleotide.

As used herein, the term “tailed random primer” refers to a single-stranded nucleic acid molecule having a 5′ nucleotide sequence (e.g., a 5′ nucleotide sequence identical or complementary to a first sequencing primer) and a 3′ nucleic acid sequence, in which the 3′ nucleotide comprises random nucleotides (e.g., from about 3 to about 15 random nucleotides, about 6 to about 12 random nucleotides). In some embodiments, the 3′ nucleotide sequence comprising random nucleotides is at least 6 nucleotides in length, e.g. 6 nucleotides or more, 7 nucleotides or more, 8 nucleotides or more, 9 nucleotides or more, 10 nucleotides or more, 11 nucleotides or more, 12 nucleotides or more, 13 nucleotides or more, 14 nucleotides or more, 15 nucleotides or more, 20 nucleotides or more, 25 nucleotides or more in length. In some embodiments, the 3′ nucleotide sequence comprising random nucleotides is 3 to 6 nucleotides in length, 3 to 9 nucleotides in length, 3 to 12 nucleotides in length, 5 to 9 nucleotides in length, 6 to 12 nucleotides in length, 3 to 25 nucleotides in length, 6 to 15 nucleotides in length, or 6 to 25 nucleotides in length.

In some embodiments, a tailed random primer can further comprise a spacer between the 5′ nucleotide sequence and the 3′ nucleotide sequence comprising about 6 to about 12 random nucleotides. In some embodiments, the spacer may be 3 to 6 nucleotides in length, 3 to 12 nucleotides in length, 3 to 25 nucleotides in length, 3 to 45 nucleotides in length, 6 to 12 nucleotides in length, 8 to 16 nucleotides in length, 6 to 25 nucleotides in length, or 6 to 45 nucleotides in length. In some embodiments, for a populations of primers, the spacer is composed of random nucleotides (e.g.,NNNNNNNNN, in which each of N is independently selected from A, G, C, and T). In some embodiments, the spacer is flanked by two common regions that are complementary. In some embodiments, a population of tailed random primers can comprise individual primers with varying 3′ sequences. In some embodiments, a population of tailed random primers can comprise individual primers with identical 5′ nucleotide sequences, e.g., they are all compatible with the same sequencing primer. In some embodiments, a population of tailed random primers can comprise individual primers with varying 5′ nucleotide sequences, e.g. an first individual primer is compatible with a first sequencing primer and a second individual primer is compatible with a second sequencing primer.

In some embodiments, methods described herein comprise an extension regimen or step (e.g. step (b)). In such embodiments, extension may proceed from one or more hybridized tailed random primers, using the nucleic acid molecules which the primers are hybridized to as templates. Extension steps are described herein. In some embodiments, one or more tailed random primers can hybridize to substantially all of the nucleic acids in a sample, many of which may not comprise a known target nucleotide sequence. Accordingly, in some embodiments, extension of random primers may occur due to hybridization with templates that do not comprise a known target nucleotide sequence.

In some embodiments, methods described herein may involve a polymerase chain reaction (PCR) amplification regimen, involving one or more amplification cycles (e.g. steps (c) and (d)). As used herein, the term “amplification regimen” refers to a process of specifically amplifying (e.g., increasing the abundance of) a nucleic acid of interest. In some embodiments, exponential amplification occur when products of a previous polymerase extension serve as templates for successive rounds of extension. In some embodiments, a PCR amplification regimen according to methods disclosed herein may comprise at least one, and in some cases at least 5 or more iterative cycles, in some embodiments each iterative cycle comprises steps of: 1) strand separation (e.g., thermal denaturation); 2) oligonucleotide primer annealing to template molecules; and 3) nucleic acid polymerase extension of the annealed primers. In should be appreciated that any suitable conditions and times involved in each of these steps may be used. In some embodiments, conditions and times selected may depend on the length, sequence content, melting temperature, secondary structural features, or other factors relating to the nucleic acid template and/or primers used in the reaction. In some embodiments, an amplification regimen according to methods described herein is performed in a thermal cycler, many of which are commercially available.

In some embodiments, a nucleic acid extension reaction, e.g. the extension step of PCR, involves the use of a nucleic acid polymerase. As used herein, the phrase “nucleic acid polymerase” refers an enzyme that catalyzes the template-dependent polymerization of nucleoside triphosphates to form primer extension products that are complementary to the template nucleic acid sequence. A nucleic acid polymerase enzyme initiates synthesis at the 3′ end of an annealed primer and proceeds in the direction toward the 5′ end of the template. Numerous nucleic acid polymerases are known in the art and commercially available. One group of nucleic acid polymerases are thermostable, i.e., they retain function after being subjected to temperatures sufficient to denature annealed strands of complementary nucleic acids, e.g. 94° C., or sometimes higher. A non-limiting example of a protocol for amplification involves using a polymerase (e.g., VeraSeq) under the following conditions: 98° C. for 30 s, following by 14-22 cycles comprising melting at 98° C. for 10 s, followed by annealing at 68° C. for 30 s, followed by extension at 72° C. 3 min, followed by holding of the reaction at 4° C. However, other appropriate reaction conditions may be used. In some embodiments, annealing/extension temperatures may be adjusted to account for differences in salt concentration (e.g., 3° C. higher to higher salt concentrations).

In some embodiments, a nucleic acid polymerase is used under conditions in which the enzyme performs a template-dependent extension. In some embodiments, the nucleic acid polymerase is DNA polymerase I, Taq polymerase, Pheonix Taq polymerase, Phusion polymerase, T4 polymerase, T7 polymerase, Klenow fragment, Klenow exo-, phi29 polymerase, AMV reverse transcriptase, M-MuLV reverse transcriptase, HIV-1 reverse transcriptase, VeraSeq ULtra polymerase, VeraSeq HF 2.0 polymerase, EnzScript or another appropriate polymerase. In some embodiments, a nucleic acid polymerase is not a reverse transcriptase. In some embodiments, a nucleic acid polymerase acts on a DNA template. In some embodiments, the nucleic acid polymerase acts on an RNA template. In some embodiments, an extension reaction involves reverse transcription performed on a RNA to produce a complementary DNA molecule (RNA-dependent DNA polymerase activity). In some embodiments, a reverse transcriptase is a mouse molony marine leukemia virus (M-MLV) polymerase, AMV reverse transcriptase, RSV reverse transcriptase, HIV-1 reverse transcriptase, HIV-2 reverse transcriptase or another appropriate reverse transcriptase.

In some embodiments, a nucleic acid amplification reaction involves cycles including a strand separation step generally involving heating of the reaction mixture. As used herein, the term “strand separation” or “separating the strands” means treatment of a nucleic acid sample such that complementary double-stranded molecules are separated into two single strands available for annealing to an oligonucleotide primer. In some embodiments, strand separation according to methods described herein is achieved by heating the nucleic acid sample above its melting temperature (Tₘ). In some embodiments, for a sample containing nucleic acid molecules in a reaction preparation suitable for a nucleic acid polymerase, heating to 94° C. is sufficient to achieve strand separation. In some embodiments, a suitable reaction preparation contains one or more salts (e.g., 1 to 100 mM KCl, 0.1 to 10 MgCl₂), at least one buffering agent (e.g., 1 to 20 mM Tris-HCL), and a carrier (e.g., 0.01 to 0.5% BSA). A non-limiting example of a suitable buffer comprises 50 mM KCl, 10 mM Tris-HCl (pH 8.8@25° C.), 0.5 to 3 mM MgCl₂, and 0.1% BSA.

In some embodiments, a nucleic acid amplification involves annealing primers to nucleic acid templates having a strands characteristic of a target nucleic acid. In some embodiments, a strand of a target nucleic acid can serve as a template nucleic acid.

As used herein, the term “anneal” refers to the formation of one or more complementary base pairs between two nucleic acids. In some embodiments, annealing involves two complementary or substantially complementary nucleic acids strands hybridizing together. In some embodiments, in the context of an extension reaction annealing involves the hybridization of primer to a template such that a primer extension substrate for a template-dependent polymerase enzyme is formed. In some embodiments, conditions for annealing (e.g., between a primer and nucleic acid template) may vary based of the length and sequence of a primer. In some embodiments, conditions for annealing are based upon a Tₘ (e.g., a calculated Tₘ) of a primer. In some embodiments, an annealing step of an extension regimen involves reducing the temperature following strand separation step to a temperature based on the Tₘ (e.g., a calculated Tₘ) for a primer, for a time sufficient to permit such annealing. In some embodiments, a Tₘ can be determined using any of a number of algorithms (e.g., OLIGO™ (Molecular Biology Insights Inc. Colorado) primer design software and VENTRO NTI™ (Invitrogen, Inc. California) primer design software and programs available on the internet, including Primer3, Oligo Calculator, and NetPrimer (Premier Biosoft; Palo Alto, Calif.; and freely available on the world wide web (e.g., at premierbiosoft.com/netprimer/netprlaunch/Help/xnetprlaunch.html). In some embodiments, the Tₘ of a primer can be calculated using following formula, which is used by NetPrimer software and is described in more detail in Frieir et al. PNAS 1986 83:9373-9377 which is incorporated by reference herein in its entirety.

Tₘ=ΔH/(ΔS+R*ln (C/4))+16.6 log([K⁺]/(1+0.7[K⁺]))−273.15

wherein, ΔH is enthalpy for helix formation; ΔS is entropy for helix formation; R is molar gas constant (1.987 cal/° C.*mol); C is the nucleic acid concentration; and [K⁺] is salt concentration. For most amplification regimens, the annealing temperature is selected to be about 5° C. below the predicted Tₘ, although temperatures closer to and above the Tₘ (e.g., between 1° C. and 5° C. below the predicted Tₘ or between 1° C. and 5° C. above the predicted Tₘ) can be used, as can, for example, temperatures more than 5° C. below the predicted Tₘ (e.g., 6° C. below, 8° C. below, 10° C. below or lower). In some embodiments, the closer an annealing temperature is to the Tₘ, the more specific is the annealing. In some embodiments, the time used for primer annealing during an extension reaction (e.g., within the context of a PCR amplification regimen) is determined based, at least in part, upon the volume of the reaction (e.g. with larger volumes involving longer times). In some embodiments, the time used for primer annealing during an extension reaction (e.g., within the context of a PCR amplification regimen) is determined based, at least in part, upon primer and template concentrations (e.g. with higher relative concentrations of primer to template involving less time than lower relative concentrations). In some embodiments, depending upon volume and relative primer/template concentration, primer annealing steps in an extension reaction (e.g., within the context of an amplification regimen) can be in the range of 1 second to 5 minutes, 10 seconds and 2 minutes, or 30 seconds to 2 minutes. As used herein, “substantially anneal” refers to an extent to which complementary base pairs form between two nucleic acids that, when used in the context of a PCR amplification regimen, is sufficient to produce a detectable level of a specifically amplified product.

As used herein, the term “polymerase extension” refers to template-dependent addition of at least one complementary nucleotide, by a nucleic acid polymerase, to the 3′ end of an primer that is anneal to a nucleic acid template. In some embodiments, polymerase extension adds more than one nucleotide, e.g., up to and including nucleotides corresponding to the full length of the template. In some embodiments, conditions for polymerase extension are based, at least in part, on the identity of the polymerase used. In some embodiments, the temperature used for polymerase extension is based upon the known activity properties of the enzyme. In some embodiments, in which annealing temperatures are below the optimal temperatures for the enzyme, it may be acceptable to use a lower extension temperature. In some embodiments, enzymes may retain at least partial activity below their optimal extension temperatures. In some embodiments, a polymerase extension (e.g., performed thermostable polymerases) (e.g., Taq polymerase and variants thereof) is performed at 65° C. to 75° C. or 68° C. to 72° C. In some embodiments, methods provided herein involve polymerase extension of primers that are anneal to nucleic acid templates at each cycle of a PCR amplification regimen. In some embodiments, a polymerase extension is performed using a polymerase that has relatively strong strand displacement activity. In some embodiments, polymerases having strong strand displacement are useful for preparing nucleic acids for purposes of detecting fusions (e.g., 5′ fusions).

In some embodiments, primer extension is performed under conditions that permit the extension of annealed oligonucleotide primers. As used herein, the term “conditions that permit the extension of an annealed oligonucleotide such that extension products are generated” refers to the set of conditions including, for example temperature, salt and co-factor concentrations, pH, and enzyme concentration under which a nucleic acid polymerase catalyzes primer extension. In some embodiments, such conditions are based, at least in part, on the nucleic acid polymerase being used. In some embodiments, a polymerase may perform a primer extension reaction in a suitable reaction preparation. In some embodiments, a suitable reaction preparation contains one or more salts (e.g., 1 to 100 mM KCl, 0,1 to 10 MgCl₂), at least one buffering agent (e.g., 1 to 20 mM Tris-HCL), a carrier (e.g., 0.01 to 0.5% BSA) and one or more NTPs (e.g., 10 to 200 uM of each of dATP, dTTP, dCTP, and dGTP). A further non-limiting set of conditions is 50 mM KCl, 10 mM Tris-HCl (pH 8.8@25° C.), 0.5 to 3 mM MgCl₂, 200 uM each dNTP, and 0.1% BSA at 72° C., under which a polymerase (e.g., Taq polymerase) catalyzes primer extension. In some embodiments, conditions for initiation and extension may include the presence of one, two, three or four different deoxyribonucleoside triphosphates (e.g., selected from dATP, dTTP, dCTP, and dGTP) and a polymerization-inducing agent such as DNA polymerase or reverse transcriptase, in a suitable buffer. In some embodiments, a “buffer” may include solvents (e.g., aqueous solvents) plus appropriate cofactors and reagents which affect pH, ionic strength, etc.).

In some embodiments, nucleic acid amplification involve up to 5, up to 10, up to 20, up to 30, up to 40 or more rounds (cycles) of amplification. In some embodiments, nucleic acid amplification may comprise a set of cycles of a PCR amplification regimen from 5 cycles to 20 cycles in length. In some embodiments, an amplification step may comprise a set of cycles of a PCR amplification regimen from 10 cycles to 20 cycles in length. In some embodiments, each amplification step can comprise a set of cycles of a PCR amplification regimen from 12 cycles to 16 cycles in length. In some embodiments, an annealing temperature can be less than 70° C. In some embodiments, an annealing temperature can be less than 72° C. In some embodiments, an annealing temperature can be about 65° C. In some embodiments, an annealing temperature can be from about 61 to about 72° C.

In various embodiments, methods and compositions described herein relate to performing a PCR amplification regimen with one or more of the types of primers described herein. As used herein, “primer” refers to an oligonucleotide capable of specifically annealing to a nucleic acid template and providing a 3′ end that serves as a substrate for a template-dependent polymerase to produce an extension product which is complementary to the template. In some embodiments, a primer useful in methods described herein is single-stranded, such that the primer and its complement can anneal to form two strands. Primers according to methods and compositions described herein may comprise a hybridization sequence (e.g., a sequence that anneals with a nucleic acid template) that is less than or equal to 300 nucleotides in length, e.g., less than or equal to 300, or 250, or 200, or 150, or 100, or 90, or 80, or 70, or 60, or 50, or 40, or 30 or fewer, or 20 or fewer, or 15 or fewer, but at least 6 nucleotides in length. In some embodiments, a hybridization sequence of a primer may be 6 to 50 nucleotides in length, 6 to 35 nucleotides in length, 6 to 20 nucleotides in length, 10 to 25 nucleotides in length.

Any suitable method may be used for synthesizing oligonucleotides and primers. In some embodiments, commercial sources offer oligonucleotide synthesis services suitable for providing primers for use in methods and compositions described herein, e.g. INVITROGEN™ Custom DNA Oligos; Life Technologies; Grand Island, N.Y. or custom DNA Oligos from IDT; Coralville, Iowa).

In some embodiments, after an extension from a tailed random primer has occurred, the extension product and template (e.g., the target nucleic acid) can be amplified in a first amplification step. In some embodiments, amplification may involve a set of PCR amplification cycles using a first target-specific primer and a first tail primer. In some embodiments, the amplification may result in at least part of the tailed random primer sequence present in the extension product being amplified. In some embodiments, the amplification may result in all of the tailed random primer sequence present in the extension product being amplified.

As used herein, the term “first target-specific primer” refers to a single-stranded oligonucleotide comprising a nucleic acid sequence that can specifically anneal under suitable annealing conditions to a nucleic acid template that has a strand characteristic of a target nucleic acid.

In some embodiments, a primer (e.g., a target specific primer) can comprise a 5′ tag sequence portion. In some embodiments, multiple primers (e.g., all first-target specific primers) present in a reaction can comprise identical 5′ tag sequence portions. In some embodiments, in a multiplex PCR reaction, different primer species can interact with each other in an off-target manner, leading to primer extension and subsequently amplification by DNA polymerase. In such embodiments, these primer dimers tend to be short, and their efficient amplification can overtake the reaction and dominate resulting in poor amplification of desired target sequence. Accordingly, in some embodiments, the inclusion of a 5′ tag sequence in primers (e.g., on target specific primer(s)) may result in formation of primer dimers that contain the same complementary tails on both ends. In some embodiments, in subsequent amplification cycles, such primer dimers would denature into single-stranded DNA primer dimers, each comprising complementary sequences on their two ends which are introduced by the 5′ tag. In some embodiments, instead of primer annealing to these single stranded DNA primer dimers, an intra-molecular hairpin (a panhandle like structure) formation may occur due to the proximate accessibility of the complementary tags on the same primer dimer molecule instead of an inter-molecular interaction with new primers on separate molecules. Accordingly, in some embodiments, these primer dimers may be inefficiently amplified, such that primers are not exponentially consumed by the dimers for amplification; rather the tagged primers can remain in high and sufficient concentration for desired specific amplification of target sequences. In some embodiments, accumulation of primer dimers may be undesirable in the context of multiplex amplification because they compete for and consume other reagents in the reaction.

In some embodiments, a 5′ tag sequence can be a GC-rich sequence. In some embodiments, a 5′ tag sequence may comprise at least 50% GC content, at least 55% GC content, at least 60% GC content, at least 65% GC content, at least 70% GC content, at least 75% GC content, at least 80% GC content, or higher GC content. In some embodiments, a tag sequence may comprise at least 60% GC content. In some embodiments, a tag sequence may comprise at least 65% GC content.

In some embodiments, a target-specific primer (e.g., a second target-specific primer) is a single-stranded oligonucleotide comprising a 3′ portion comprising a nucleic acid sequence that can specifically anneal to a portion of a known target nucleotide sequence of an amplicon of an amplification reaction, and a 5′ portion comprising a tag sequence (e.g., a nucleotide sequence that is identical to or complementary to a sequencing primer (e.g., a second sequencing primer). In some embodiments, a second target-specific primer” is a single-stranded oligonucleotide comprising a 3′ portion comprising a nucleic acid sequence that can specifically anneal to a portion of the known target nucleotide sequence comprised by the amplicon resulting from step (c), and a 5′ portion comprising a nucleic acid sequence that is identical to or complementary to a sequencing primer (e.g., a second sequencing primer).

In some embodiments, a second target-specific primer of an amplification regimen is nested with respect to a first target-specific primer of the amplification regimen. In some embodiments, the second target-specific primer is nested with respect to the first target-specific primer by at least 3 nucleotides, e.g. by 3 or more, 4 or more, 5 or more, 6 or more, 7 or more, 8 or more, 9 or more, 10 or more, or 15 or more nucleotides. In some embodiments, all of the target-specific primers (e.g., second target-specific primers) used in an amplification regimen comprise the same 5′ portion. In some embodiments, the 5′ portion target-specific primer can be configured to suppress primer dimers as described herein.

In some embodiments, first and second target-specific primers are used in an amplification regimen that are substantially complementary to the same strand of a target nucleic acid. In some embodiments, portions of the first and second target-specific primers that specifically anneal to a target sequence (e.g., a known target sequence) can comprise a total of at least 20 unique bases of the known target nucleotide sequence, e.g. 20 or more unique bases, 25 or more unique bases, 30 or more unique bases, 35 or more unique bases, 40 or more unique bases, or 50 or more unique bases. In some embodiments, portions of first and second target-specific primers that specifically anneal to a target sequence (e.g., a known target sequence) can comprise a total of at least 30 unique bases of the known target nucleotide sequence.

As used herein, the term “first tail primer” refers to a nucleic acid molecule comprising a nucleic acid sequence identical to the tail portion of tailed primer.

As used herein, the term “second tail primer” refers to a nucleic acid molecule comprising a nucleic acid sequence identical to a portion of a first sequencing primer, adapter, index primer, etc. and is optionally nested with respect to a first tailed primer. In some embodiments, the second tail primer sits outside of the first tail primer to facilitate addition of appropriate index tags, adapters (e.g., for use in a sequencing platform), etc. In some embodiments, a second tailed primer is identical to a sequencing primer. In some embodiments, a second tailed primer is complementary to a sequencing primer.

In some embodiments, a second tail primer is nested with respect to a first tail primer. In some embodiments, a second tail primer is not nested with respect to a first tail primer. In some embodiments, tail primers of an amplification regimen are nested with respect to one another by at least 3 nucleotides, e.g. by 3 nucleotides, by 4 nucleotides, by 5 nucleotides, by 6 nucleotides, by 7 nucleotides, by 8 nucleotides, by 9 nucleotides, by 10 nucleotides or more.

In some embodiments, a first tail primer comprises a nucleic acid sequence identical to or complementary to the extension product of step (b) strand which is not comprised by the second tail primer and which is located closer to the 5′ end of the tailed random primer than any of the sequence identical to or complementary to the second tail primer. Thus, in some embodiments, a second tail primer sits outside of a region added by a random tail primer (5′ end), e.g., within the 5′ tail added by the first tail primers.

In some embodiments, a first tail primer can comprise a nucleic acid sequence identical to or complementary to a stretch (e.g., of about 20 nucleotides) of the 5′-most nucleotides of a tailed random primer, and a second tail primer can comprise a nucleic acid sequence identical to or complementary to about 30 bases of a tailed random primer, with a 5′ nucleotides that is at least 3 nucleotides 3′ of the 5′ terminus of the tailed random primer.

In some embodiments, use of nested tail primers minimizes or eliminates the production of final amplicons that are amplifiable (e.g. during bridge PCR or emulsion PCR) but cannot be sequenced, a situation that can arise during hemi-nested methods. In some embodiments, hemi-nested approaches using a primer identical to a sequencing primer can result in the carry-over of undesired amplification products from a first PCR step to a second PCR step and may yield artificial sequencing reads. In some embodiments, the use of two tail primers, as described herein can reduce, and in some embodiments eliminate, these problems.

In some embodiments, in a first PCR amplification cycle of a first amplification step, a first target-specific primer can specifically anneal to a template strand of any nucleic acid comprising the known target nucleotide sequence. In some embodiments, depending upon the orientation with which the first target-specific primer was designed, sequence upstream or downstream of the known target nucleotide sequence, and complementary to the template strand will be synthesized. In some embodiments, in which an extension product is formed that comprises the hybridization sequence with which the first target-specific primer forms complementary base pairs, a double-stranded amplification product can be formed that comprises the first target-specific primer (and the sequence complementary thereto), the target nucleotide sequence downstream of the first target-specific primer (and the sequence complementary thereto), and the tailed random primer sequence (and the sequence complementary thereto). In such embodiments, in subsequent PCR amplification cycles, both the first target-specific primer and the first tail primer are capable of specifically annealing to appropriate strands of the amplification product and the sequence between the known nucleotide target sequence and the tailed random primer can be amplified.

In some embodiments, of methods described herein, a portion of an amplified product (an amplicon) is amplified in further rounds of amplification (e.g. step (d). In some embodiments, the further rounds of amplification may involve PCR amplification cycles performed using a second target-specific primer and a first sequencing primer or a second tail primer. In some embodiments, a PCR amplification cycles may involve the use of PCR parameters identical to, or which differ from, those of one or moreother (e.g., prior) of PCR amplification cycles. In some embodiments, PCR amplification regimens can have the same or different annealing temperatures or the same or different extension step time lengths.

In some embodiments, methods described herein allow for determining the nucleotide sequence contiguous to a known target nucleotide sequence on either or both flanking regions of the known target nucleotide sequence. Regardless of whether the target nucleic acid normally exists as a single-stranded or double-stranded nucleic acid, sequence information may be represented in a single-stranded format (Strand A), from 5′ to 3′. In some embodiments, if the sequence 5′ to a known target nucleotide sequence of Strand A is to be determined, gene-specific primers can be complementary to (anneal to) Strand A. If the sequence 3′ to a known target nucleotide sequence of Strand A is to be determined, the gene-specific primers can be identical to Strand A, such that they will anneal to the complementary strand of a double-stranded target nucleic acid.

In some embodiments, methods described herein, relating to the use of a first and second gene-specific primer can result in assays with a superior on-target rate, e.g. 70-90%. In some embodiments, the assays and methods described herein can have a target specificity rate of at least 85%.

In some embodiments, primers disclosed herein (e.g., target-specific primers, tail primers) are designed such that they will specifically anneal to their complementary sequences at an annealing temperature of from about 61 to 72° C., e.g. from about 61 to 69° C., from about 63 to 69° C., from about 63 to 67° C., from about 64 to 66° C. In some embodiments, primers disclosed herein are designed such that they will specifically anneal to their complementary sequences at an annealing temperature of less than 72° C. In some embodiments, primers disclosed herein are designed such that they will specifically anneal to their complementary sequences at an annealing temperature of less than 70° C. In some embodiments, primers disclosed herein are designed such that they will specifically anneal to their complementary sequences at an annealing temperature of less than 68° C. In some embodiments, primers disclosed herein are designed such that they will specifically anneal to their complementary sequences at an annealing temperature of about 65° C.

In some embodiments, portions of the target-specific primers that specifically anneal to the known target nucleotide sequence will anneal specifically at a temperature of about 61 to 72° C., e.g. from about 61 to 69° C., from about 63 to 69° C., from about 63 to 67° C., from about 64 to 66° C. In some embodiments, portions of the target-specific primers that specifically anneal to the known target nucleotide sequence will anneal specifically at a temperature of about 65° C. in a PCR buffer.

In some embodiments, primers described herein do not comprise modified bases (e.g. the primers can not comprise a blocking 3′ amine). However, in some embodiments, primers described herein do comprise modified or non-naturally occurring bases. In some embodiments, primers may be modified with a label capable of providing a detectable signal, either directly or indirectly. Non-limiting examples of such labels include radioisotopes, fluorescent molecules, biotin, and others. In some embodiments, primers disclosed herein may include contain a biotin linker or other suitable linker (e.g., for conjugating the primer to a support). In some embodiments, primer may contain a target sequence of an endonucleases such that cleavage with the appropriate enzyme. In other embodiments, the 5′ end of a primer may include a sequence that is complementary with a nucleic acid bound to a bead or other support, e.g., a flow cell substrate. Primers may or may not comprise modified internucleoside linkages.

In some embodiments, of methods described herein, nucleic acids (e.g., amplified nucleic acids, extension products, target nucleic acids) can be sequenced, e.g. the nucleic acids resulting from step (d) can be sequenced. In some embodiments, sequencing can be performed by a next-generation sequencing method. As used herein “next-generation sequencing” refers to oligonucleotide sequencing technologies that have the capacity to sequence oligonucleotides at speeds above those possible with conventional sequencing methods (e.g. Sanger sequencing), due to performing and reading out thousands to millions of sequencing reactions in parallel. Non-limiting examples of next-generation sequencing methods/platforms include Massively Parallel Signature Sequencing (Lynx Therapeutics); 454 pyro-sequencing (454 Life Sciences/Roche Diagnostics); solid-phase, reversible dye-terminator sequencing (Solexa/Illumina): SOLiD technology (Applied Biosystems); Ion semiconductor sequencing (ION Torrent); DNA nanoball sequencing (Complete Genomics); and technologies available from Pacific Biosciences, Intelligen Bio-systems, Oxford Nanopore Technologies, and Helicos Biosciences. In some embodiments, the sequencing primers can comprise portions compatible with the selected next-generation sequencing method. Next-generation sequencing technologies and the constraints and design parameters of associated sequencing primers are well known in the art (see, e.g. Shendure, et al., “Next-generation DNA sequencing,” Nature, 2008, vol. 26, No. 10, 1135-1145; Mardis, “The impact of next-generation sequencing technology on genetics,” Trends in Genetics, 2007, vol. 24, No. 3, pp. 133-141; Su, et al., “Next-generation sequencing and its applications in molecular diagnostics” Expert Rev Mol Diagn, 2011, 11(3):333-43; Zhang et al., “The impact of next-generation sequencing on genomics”, J Genet Genomics, 2011, 38(3):95-109; (Nyren, P. et al. Anal Biochem 208: 17175 (1993); Bentley, D. R. Curr Opin Genet Dev 16:545-52 (2006); Strausberg, R. L., et al. Drug Disc Today 13:569-77 (2008); U.S. Pat. No. 7,282,337; U.S. Pat. No. 7,279,563; U.S. Pat. No. 7,226,720; U.S. Pat. No. 7,220,549; U.S. Pat. No. 7,169,560; U.S. Pat. No. 6,818,395; U.S. Pat. No. 6,911,345; US Pub. Nos. 2006/0252077; 2007/0070349; and 20070070349; which are incorporated by reference herein in their entireties).

In some embodiments, the sequencing step involves the use of a first and second sequencing primers. In some embodiments, the first and second sequencing primers are selected to be compatible with a next-generation sequencing method as described herein.

Methods of aligning sequencing reads to known sequence databases of genomic and/or cDNA sequences are well known in the art and software is commercially available for this process. In some embodiments, reads (less the sequencing primer nucleotide sequence) which do not map, in their entirety, to wild-type sequence databases can be genomic rearrangements or large indel mutations. In some embodiments, reads (less the sequencing primer nucleotide sequence) comprising sequences which map to multiple locations in the genome can be genomic rearrangements.

In some embodiments, primers may contain additional sequences such as sequencing primer hybridization sequences (e.g., Rd1), and adapter sequences. In some embodiments the adapter sequences are sequences used with a next generation sequencing system. In some embodiments, the adapter sequences are P5 and P7 sequences for Illumina-based sequencing technology. In some embodiments, the adapter sequences are P1 and A compatible with Ion Torrent sequencing technology.

In some embodiments, when a population of tailed random primers is used in accordance with methods described herein, multiple distinguishable amplification products can be present after amplification, e.g., after step (d). In some embodiments, because tailed random primers hybridize at various positions throughout nucleic acid molecules of a sample, a set of target-specific primers can hybridize (and amplify) the extension products created by more than 1 hybridization event, e.g. one tailed random primer may hybridize at a first distance (e.g., 100 nucleotides) from a target-specific primer hybridization site, and another tailed random primer can hybridize at a second distance (e.g., 200 nucleotides) from a target-specific primer hybridization site, thereby resulting in two amplification products (e.g., a ˜100 bp amplification product and a ˜200 bp amplification product). In some embodiments, these multiple amplification products can each be sequenced in. In some embodiments, sequencing of these multiple amplification products is advantageous because it provides multiple overlapping sequence reads that can be compared with one another to detect sequence errors introduced during amplification or sequencing processes. In some embodiments, individual amplification products can be aligned and where they differ in the sequence present at a particular base, an artifact or error of PCR and/or sequencing may be present.

In some embodiments, target nucleic acids and/or amplification products thereof can be isolated from enzymes, primers, or buffer components before and/or after any of appropriate step of a method. Any suitable methods for isolating nucleic acids may be used. In some embodiments, the isolation can comprise Solid Phase Reversible Immobilization (SPRI) cleanup. Methods for SPRI cleanup are well known in the art and kits are commercially available, e.g.

Agencourt AMPure XP-PCR Purification (Cat No. A63880, Beckman Coulter; Brea, Calif.). In some embodiments, enzymes can be inactivated by heat treatment.

In some embodiments, unhybridized primers can be removed from a nucleic acid preparation using appropriate methods (e.g., purification, digestion, etc.). In some embodiments, a nuclease (e.g., exonuclease I) is used to remove primer from a preparation. In some embodiments, such nucleases are heat inactivated subsequent to primer digestion. Once the nucleases are inactivated a further set of primers may be added together with other appropriate components (e.g., enzymes, buffers) to perform a further amplification reaction.

In some embodiments, a target nucleic acid genomic DNA or a portion thereof. In some embodiments, a target nucleic acid can be ribonucleic acid (RNA), e.g. mRNA, or a portion thereof. In some embodiments, a target nucleic acid can be a cDNA or a portion thereof.

In some embodiments, the sample comprises single-stranded cDNA, e.g. at least 10% of the cDNA is single-stranded, e.g. 10% or more, 20% or more, 30% or more, 40% or more, 50% or more, 60% or more, 70% or more, 80% or more, 90% or more, or 95% or more of the cDNA is single-stranded.

In some embodiments, the sample comprises single-stranded gDNA, e.g. at least 10% of the gDNA is single-stranded, e.g. 10% or more, 20% or more, 30% or more, 40% or more, 50% or more, 60% or more, 70% or more, 80% or more, 90% or more, or 95% or more of the gDNA is single-stranded.

Many of the sequencing methods suitable for use in methods described herein provide sequencing runs with optimal read lengths of tens to hundreds of nucleotide bases (e.g. Ion Torrent technology can produce read lengths of 200-400 bp). Target nucleic acids may or may not be substantially longer than this optimal read length. In some embodiments, in order for an amplified nucleic acid portion (e.g. the portion resulting from step (d)) to be of a suitable length for use in a particular sequencing technology, the average distance between the known target nucleotide sequence and an end of the target nucleic acid to which a tailed random primer is hybridizable should be as close to the optimal read length of the selected technology as possible. In some embodiments, if the optimal read-length of a given sequencing technology is 200 bp, then the nucleic acid molecules amplified in accordance with methods described herein should have an average length of about 800 bp, about 700 bp, about 600 bp, about 500 bp, about 400 bp, about 300 bp, about 200 bp or less.

Nucleic acids used herein (e.g., target nucleic acids prior to sequencing) can be sheared, e.g. mechanically or enzymatically sheared, to generate fragments of any desired size. Non-limiting examples of mechanical shearing processes include sonication, nebulization, and AFA™ shearing technology available from Covaris (Woburn, Mass.). In some embodiments, a nucleic acid can be mechanically sheared by sonication.

In some embodiments, a target nucleic acid is not sheared or digested. In some embodiments, nucleic acid products of preparative steps (e.g., extension products, amplification products) are not sheared or enzymatically digested.

In some embodiments, when a target nucleic acid an RNA, the sample can be subjected to a reverse transcriptase regimen to generate DNA template and the DNA template can then be sheared. In some embodiments, target RNA can be sheared before performing a reverse transcriptase regimen. In some embodiments, a sample comprising target RNA can be used in methods described herein using total nucleic acids extracted from either fresh or degraded specimens; without the need of genomic DNA removal for cDNA sequencing; without the need of ribosomal RNA depletion for cDNA sequencing; without the need of mechanical or enzymatic shearing in any of the steps; by subjecting the RNA for double-stranded cDNA synthesis using random hexamers.

In some embodiments, a known target nucleic acid can contain a fusion sequence resulting from a gene rearrangement. In some embodiments, methods described herein are suited for determining the presence and/or identity of a gene rearrangement. In some embodiments, identity of one portion of a gene rearrangement is previously known (e.g., the portion of a gene rearrangement that is to be targeted by the gene-specific primers) and the sequence of the other portion may be determined using methods disclosed herein. In some embodiments, a gene rearrangement can involve an oncogene. In some embodiments, a gene rearrangement can comprise a fusion oncogene.

In some embodiments, a target nucleic acid is present in or obtained from an appropriate sample (e.g., a food sample, environmental sample, biological sample e.g., blood sample, etc.). In some embodiments, the sample is a biological sample obtained from a subject. In some embodiments a sample can be a diagnostic sample obtained from a subject. In some embodiments, a sample can further comprise proteins, cells, fluids, biological fluids, preservatives, and/or other substances. By way of non-limiting example, a sample can be a cheek swab, blood, serum, plasma, sputum, cerebrospinal fluid, urine, tears, alveolar isolates, pleural fluid, pericardial fluid, cyst fluid, tumor tissue, tissue, a biopsy, saliva, an aspirate, or combinations thereof. In some embodiments, a sample can be obtained by resection or biopsy.

In some embodiments, the sample can be obtained from a subject in need of treatment for a disease associated with a genetic alteration, e.g. cancer or a hereditary disease. In some embodiments, a known target sequence is present in a disease-associated gene.

In some embodiments, a sample is obtained from a subject in need of treatment for cancer. In some embodiments, the sample comprises a population of tumor cells, e.g. at least one tumor cell. In some embodiments, the sample comprises a tumor biopsy, including but not limited to, untreated biopsy tissue or treated biopsy tissue (e.g. formalin-fixed and/or paraffin-embedded biopsy tissue).

In some embodiments, the sample is freshly collected. In some embodiments, the sample is stored prior to being used in methods and compositions described herein. In some embodiments, the sample is an untreated sample. As used herein, “untreated sample” refers to a biological sample that has not had any prior sample pre-treatment except for dilution and/or suspension in a solution. In some embodiments, a sample is obtained from a subject and preserved or processed prior to being utilized in methods and compositions described herein. By way of non-limiting example, a sample can be embedded in paraffin wax, refrigerated, or frozen. A frozen sample can be thawed before determining the presence of a nucleic acid according to methods and compositions described herein. In some embodiments, the sample can be a processed or treated sample. Exemplary methods for treating or processing a sample include, but are not limited to, centrifugation, filtration, sonication, homogenization, heating, freezing and thawing, contacting with a preservative (e.g. anti-coagulant or nuclease inhibitor) and any combination thereof. In some embodiments, a sample can be treated with a chemical and/or biological reagent. Chemical and/or biological reagents can be employed to protect and/or maintain the stability of the sample or nucleic acid comprised by the sample during processing and/or storage. In addition, or alternatively, chemical and/or biological reagents can be employed to release nucleic acids from other components of the sample. By way of non-limiting example, a blood sample can be treated with an anti-coagulant prior to being utilized in methods and compositions described herein. Suitable methods and processes for processing, preservation, or treatment of samples for nucleic acid analysis may be used in the method disclosed herein. In some embodiments, a sample can be a clarified fluid sample, for example, by centrifugation. In some embodiments, a sample can be clarified by low-speed centrifugation (e.g. 3,000×g or less) and collection of the supernatant comprising the clarified fluid sample.

In some embodiments, a nucleic acid present in a sample can be isolated, enriched, or purified prior to being utilized in methods and compositions described herein. Suitable methods of isolating, enriching, or purifying nucleic acids from a sample may be used. For example, kits for isolation of genomic DNA from various sample types are commercially available (e.g. Catalog Nos. 51104, 51304, 56504, and 56404; Qiagen; Germantown, Md.). In some embodiments, methods described herein relate to methods of enriching for target nucleic acids, e.g., prior to a sequencing of the target nucleic acids. In some embodiments, a sequence of one end of the target nucleic acid to be enriched is not known prior to sequencing. In some embodiments, methods described herein relate to methods of enriching specific nucleotide sequences prior to determining the nucleotide sequence using a next-generation sequencing technology. In some embodiments, methods of enriching specific nucleotide sequences do not comprise hybridization enrichment.

Methods described herein can be employed in a multiplex format. In embodiments of methods described herein, multiplex applications can include determining the nucleotide sequence contiguous to one or more known target nucleotide sequences. As used herein, “multiplex amplification” refers to a process involve simultaneous amplification of more than one target nucleic acid in one reaction vessel. In some embodiments, methods involve subsequent determination of the sequence of the multiplex amplification products using one or more sets of primers. Multiplex can refer to the detection of between about 2-1,000 different target sequences in a single reaction. As used herein, multiplex refers to the detection of any range between 2-1,000, e.g., between 5-500, 25-1000, or 10-100 different target sequences in a single reaction, etc. The term “multiplex” as applied to PCR implies that there are primers specific for at least two different target sequences in the same PCR reaction.

In some embodiments, target nucleic acids in a sample, or separate portions of a sample, can be amplified with a plurality of primers (e.g., a plurality of first and second target-specific primers). In some embodiments, the plurality of primers (e.g., a plurality of first and second target-specific primers) can be present in a single reaction mixture, e.g. multiple amplification products can be produced in the same reaction mixture. In some embodiments, the plurality of primers (e.g., a plurality of sets of first and second target-specific primers) can specifically anneal to known target sequences comprised by separate genes. In some embodiments, at least two sets of primers (e.g., at least two sets of first and second target-specific primers) can specifically anneal to different portions of a known target sequence. In some embodiments, at least two sets of primers (e.g., at least two sets of first and second target-specific primers) can specifically anneal to different portions of a known target sequence comprised by a single gene. In some embodiments, at least two sets of primers (e.g., at least two sets of first and second target-specific primers) can specifically anneal to different exons of a gene comprising a known target sequence. In some embodiments, the plurality of primers (e.g., first target-specific primers) can comprise identical 5′ tag sequence portions.

In embodiments of methods described herein, multiplex applications can include determining the nucleotide sequence contiguous to one or more known target nucleotide sequences in multiple samples in one sequencing reaction or sequencing run. In some embodiments, multiple samples can be of different origins, e.g. from different tissues and/or different subjects. In such embodiments, primers (e.g., tailed random primers) can further comprise a barcode portion. In some embodiments, a primer (e.g., a tailed random primer) with a unique barcode portion can be added to each sample and ligated to the nucleic acids therein; the samples can subsequently be pooled.

In some embodiments of methods described herein, a determination of the sequence contiguous to a known oligonucleotide target sequence can provide information relevant to treatment of disease. Thus, in some embodiments, methods disclosed herein can be used to aid in treating disease. In some embodiments, a sample can be from a subject in need of treatment for a disease associated with a genetic alteration. In some embodiments, a known target sequence a sequence of a disease-associated gene, e.g. an oncogene. In some embodiments, a sequence contiguous to a known oligonucleotide target sequence and/or the known oligonucleotide target sequence can comprise a mutation or genetic abnormality which is disease-associated, e.g. a SNP, an insertion, a deletion, and/or a gene rearrangement. In some embodiments, a sequence contiguous to a known target sequence and/or a known target sequence present in a sample comprised sequence of a gene rearrangement product. In some embodiments, a gene rearrangement can be an oncogene, e.g. a fusion oncogene.

Certain treatments for cancer are particularly effective against tumors comprising certain oncogenes, e.g. a treatment agent which targets the action or expression of a given fusion oncogene can be effective against tumors comprising that fusion oncogene but not against tumors lacking the fusion oncogene. Methods described herein can facilitate a determination of specific sequences that reveal oncogene status (e.g. mutations, SNPs, and/or rearrangements). In some embodiments, methods described herein can further allow the determination of specific sequences when the sequence of a flanking region is known, e.g. methods described herein can determine the presence and identity of gene rearrangements involving known genes (e.g., oncogenes) in which the precise location and/or rearrangement partner are not known before methods described herein are performed.

In some embodiments, technology described herein relates to a method of treating cancer. Accordingly, in some embodiments, methods provided herein may involve detecting, in a tumor sample obtained from a subject in need of treatment for cancer, the presence of one or more oncogene rearrangements; and administering a cancer treatment which is effective against tumors having any of the detected oncogene rearrangements. In some embodiments, technology described herein relates to a method of determining if a subject in need of treatment for cancer will be responsive to a given treatment. Accordingly, in some embodiments, methods provided herein may involve detecting, in a tumor sample obtained from a subject, the presence of an oncogene rearrangement, in which the subject is determined to be responsive to a treatment targeting an oncogene rearrangement product if the presence of the oncogene rearrangement is detected.

In some embodiments, a subject is in need of treatment for lung cancer. In some embodiments, e.g. when the sample is obtained from a subject in need of treatment for lung cancer, the known target sequence can comprise a sequence from a gene selected from the group of ALK, ROS1, and RET. Accordingly, in some embodiments, gene rearrangements result in fusions involving the ALK, ROS1, or RET. Non-limiting examples of gene arrangements involving ALK, ROS1, or RET are described in, e.g., Soda et al. Nature 2007 448561-6: Rikova et al. Cell 2007 131:1190-1203; Kohno et al. Nature Medicine 2012 18:375-7; Takouchi et al. Nature Medicine 2012 18:378-81; which are incorporated by reference herein in their entireties. However, it should be appreciated that the precise location of a gene rearrangement, and the identity of the second gene involved in the rearrangement may not be known in advance. Accordingly, in methods described herein, the presence and identity of such rearrangements can be detected without having to know the location of the rearrangement or the identity of the second gene involved in the gene rearrangement.

In some embodiments, the known target sequence can comprise sequence from a gene selected from the group of: ALK, ROS1, and RET.

In some embodiments, the presence of a gene rearrangement of ALK in a sample obtained from a tumor in a subject can indicate that the tumor is susceptible to treatment with a treatment selected from the group consisting of: an ALK inhibitor; crizotinib (PF-02341066); AP26113; LDK378; 3-39; AF802; IPI-504; ASP3026; AP-26113; X-396; GSK-1838705A; CH5424802; diamino and aminopyrimidine inhibitors of ALK kinase activity such as NVP-TAE684 and PF-02341066 (see, e.g. Galkin et al, Proc Natl Acad Sci USA, 2007, 104:270-275; Zou et al. Cancer Res, 2007, 67: 4408-4417; Hallberg and Palmer F1000 Med Reports 2011 3:21: and Sakamoto et al. Cancer Cell 2011 19:679-690) and molecules disclosed in WO 04/079326. All of the foregoing references are incorporated by reference herein in their entireties. An ALK inhibitor can include any agent that reduces the expression and/or kinase activity of ALK or a portion thereof, including, e.g. oligonucleotides, small molecules, and/or peptides that reduce the expression and/or activity of ALK or a portion thereof. As used herein “anaplastic lymphoma kinase” or “ALK” refers to a transmembrane tyROS1ine kinase typically involved in neuronal regulation in the wildtype form. The nucleotide sequence of the ALK gene and mRNA are known for a number of species, including human (e.g. SEQ ID NO: 2 (mRNA), NCBI Gene ID: 238).

In some embodiments, the presence of a gene rearrangement of ROS1 in a sample obtained from a tumor in a subject can indicate that the tumor is susceptible to treatment with a treatment selected from the group consisting of: a ROS1 inhibitor and an ALK inhibitor as described herein above (e.g. crizotinib). A ROS1 inhibitor can include any agent that reduces the expression and/or kinase activity of ROS1 or a portion thereof, including, e.g. oligonucleotides, small molecules, and/or peptides that reduce the expression and/or activity of ROS1 or a portion thereof. As used herein “c-ros oncogene 1” or “ROS1” (also referred to in the art as ros-1) refers to a transmembrane tyrosine kinase of the sevenless subfamily and which interacts with PTPN6. Nucleotide sequences of the ROS1 gene and mRNA are known for a number of species, including human (e.g. SEQ ID NO: 1 (mRNA), NCBI Gene ID: 238).

In some embodiments, the presence of a gene rearrangement of RET in a sample obtained from a tumor in a subject can indicate that the tumor is susceptible to treatment with a treatment selected from the group consisting of: a RET inhibitor; DP-2490, DP-3636, SU5416; BAY 43-9006, BAY 73-4506 (regorafenib), ZD6474, NVP-AST487, sorafenib, RPI-1, XL184, vandetanib, sunitinib, imatinib, pazopanib, axitinib, motesanib, gefitinib, and withaferin A (see, e.g. Samadi et al. Surgery 2010 148:1228-36; Cuccuru et al. JNCI 2004 13:1006-1014; Akeno-Stuart et al. Cancer Research 2007 67:6956; Grazma et al. J Clin Oncol 2010 28:15s 5559; Mologni et al. J Mol Endocrinol 2006 37:199-212; Calmomagno et al. Journal NCI 2006 98:326-334; Mologni. Curr Med Chem 2011 18:162-175 and the compounds disclosed in WO 06/034833; US Patent Publication 2011/0201598 and U.S. Pat. No. 8,067,434). All of the foregoing references are incorporated by reference herein in their entireties. A RET inhibitor can include any agent that reduces the expression and/or kinase activity of RET or a portion thereof, including, e.g. oligonucleotides, small molecules, and/or peptides that reduce the expression and/or activity of RET or a portion thereof. As used herein “rearranged during transfection” or “RET” refers to a receptor tyrosine kinase of the cadherein superfamily which is involved in neural crest development and recognizes glial cell line-derived neurotrophic factor family signaling molecules. Nucleotide sequences of the ROS1 gene and mRNA are known for a number of species, including human (e.g. SEQ ID NOs: 3-4 (mRNA), NCBI Gene ID: 5979).

Further non-limiting examples of applications of methods described herein include detection of hematological malignancy markers and panels thereof (e.g. including those to detect genomic rearrangements in lymphomas and leukemias), detection of sarcoma-related genomic rearrangements and panels thereof; and detection of IGH/TCR gene rearrangements and panels thereof for lymphoma testing.

In some embodiments, methods described herein relate to treating a subject having or diagnosed as having, e.g. cancer with a treatment for cancer. Subjects having cancer can be identified by a physician using current methods of diagnosing cancer. For example, symptoms and/or complications of lung cancer which characterize these conditions and aid in diagnosis are well known in the art and include but are not limited to, weak breathing, swollen lymph nodes above the collarbone, abnormal sounds in the lungs, dullness when the chest is tapped, and chest pain. Tests that may aid in a diagnosis of, e.g. lung cancer include, but are not limited to, x-rays, blood tests for high levels of certain substances (e.g. calcium), CT scans, and tumor biopsy. A family history of lung cancer, or exposure to risk factors for lung cancer (e.g. smoking or exposure to smoke and/or air pollution) can also aid in determining if a subject is likely to have lung cancer or in making a diagnosis of lung cancer.

Cancer can include, but is not limited to, carcinoma, including adenocarcinoma, lymphoma, blastoma, melanoma, sarcoma, leukemia, squamous cell cancer, small-cell lung cancer, non-small cell lung cancer, gastrointestinal cancer, Hodgkin's and non Hodgkin's lymphoma, pancreatic cancer, glioblastoma, basal cell carcinoma, biliary tract cancer, bladder cancer, brain cancer including glioblastomas and medulloblastomas; breast cancer, cervical cancer, choriocarcinoma; colon cancer, colorectal cancer, endometrial carcinoma, endometrial cancer; esophageal cancer, gastric cancer; various types of head and neck cancers, intraepithelial neoplasms including Bowen's disease and Paget's disease; hematological neoplasms including acute lymphocytic and myelogenous leukemia; Kaposi's sarcoma, hairy cell leukemia; chromic myelogenous leukemia, AIDS-associated leukemias and adult T-cell leukemia lymphoma; kidney cancer such as renal cell carcinoma, T-cell acute lymphoblastic leukemia/lymphoma, lymphomas including Hodgkin's disease and lymphocytic lymphomas; liver cancer such as hepatic carcinoma and hepatoma, Merkel cell carcinoma, melanoma, multiple myeloma; neuroblastomas; oral cancer including squamous cell carcinoma; ovarian cancer including those arising from epithelial cells, sarcomas including leiomyosarcoma, rhabdomyosarcoma, liposarcoma, fibROS1arcoma, and osteosarcoma; pancreatic cancer; skin cancer including melanoma, stromal cells, germ cells and mesenchymal cells; pROS1tate cancer, rectal cancer; vulval cancer, renal cancer including adenocarcinoma; testicular cancer including germinal tumors such as seminoma, non-seminoma (teratomas, choriocarcinomas), stromal tumors, and germ cell tumors; thyroid cancer including thyroid adenocarcinoma and medullar carcinoma; esophageal cancer, salivary gland carcinoma, and Wilms' tumors. In some embodiments, the cancer can be lung cancer.

In some embodiments, methods described herein comprise administering an effective amount of compositions described herein, e.g. a treatment for cancer to a subject in order to alleviate a symptom of a cancer. As used herein, “alleviating a symptom of a cancer” is ameliorating any condition or symptom associated with the cancer. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, topical, injection, or intratumoral administration. Administration can be local or systemic. The term “effective amount” as used herein refers to the amount of a treatment needed to alleviate at least one or more symptom of the disease or disorder, and relates to a sufficient amount of pharmacological composition to provide the desired effect. The term “therapeutically effective amount” therefore refers to an amount that is sufficient to effect a particular anti-cancer effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation. The effects of any particular dosage can be monitored by a suitable bioassay. The dosage can be determined by a physician and adjusted, as appropriate, to suit observed effects of the treatment.

Non-limiting examples of a treatment for cancer can include radiation therapy, surgery, gemcitabine, cisplastin, paclitaxel, carboplatin, bortezomib, AMG479, vorinostat, rituximab, temozolomide, rapamycin, ABT-737, PI-103; alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1 and calicheamicin omega1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE.RTM. vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb®); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above. In addition, methods of treatment can further include the use of radiation or radiation therapy. Further, methods of treatment can further include the use of surgical treatments.

In some embodiments, methods described herein can be applicable for resequencing, e.g. for confirming particularly relevant, low-quality, and/or complex sequences obtained by non-directed sequencing of a large amount of nucleic acids. By way of non-limiting examples, methods described herein can allow the directed and/or targeted resequencing of targeted disease gene panels (e.g. 10-100 genes), resequencing to confirm variants obtained in large scale sequencing projects, whole exome resequencing, and/or targeted resequencing for detection of single nucleotide variants, multiple nucleotide variants, insertions, deletions, copy number changes, and methylation status.

In some embodiments, methods described herein can allow microbiota sequencing, ancient sample sequencing, and/or new variant virus genotyping.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein generally to mean a decrease by a statistically significant amount. However, for avoidance of doubt, “reduced”, “reduction”, “decrease”, or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level or non-detectable level as compared to a reference level), or any decrease between 10-100% as compared to a reference level. In the context of a marker or symptom is meant a statistically significant decrease in such level. The decrease can be, for example, at least 10%, at least 20%, at least 30%, at least 40% or more, and is preferably down to a level accepted as within the range of normal for an individual without such disorder.

The terms “increased” ,“increase”, “enhance”, or “activate” are all used herein to generally mean an increase by a statically significant amount; for the avoidance of doubt, the terms “increased”, “increase”, “enhance”, or “activate” mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of, e.g. lung cancer. A subject can be male or female.

A subject can be one who has been previously diagnosed with or identified as suffering from or having a condition in need of treatment (e.g. cancer) or one or more complications related to such a condition, and optionally, have already undergone treatment for the condition or the one or more complications related to the condition. Alternatively, a subject can also be one who has not been previously diagnosed as having the condition (e.g. cancer) or one or more complications related to the condition. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

As used herein, a “disease associated with a genetic alteration” refers to any disease which is caused by, at least in part, by an alteration in the genetic material of the subject as compared to a healthy wildtype subject, e.g. a deletion, an insertion, a SNP, a gene rearrangement. A disease can be caused by, at least in part, an alteration in the genetic material of the subject if the alteration increases the risk of the subject developing the disease, increases the subject's susceptibility to a disease (including infectious diseases, or diseases with an infectious component), causes the production of a disease-associated molecule, or causes cells to become diseased or abnormal (e.g. loss of cell cycle regulation in cancer cells). Diseases can be associated with multiple genetic alterations, e.g. cancers.

As used herein, the term “nucleic acid” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one strand nucleic acid of a denatured double-stranded DNA. Alternatively, it can be a single-stranded nucleic acid not derived from any double-stranded DNA. In one aspect, the template nucleic acid is DNA. In another aspect, the template is RNA. Suitable nucleic acid molecules are DNA, including genomic DNA or cDNA. Other suitable nucleic acid molecules are RNA, including mRNA.

The term “isolated” or “partially purified” as used herein refers, in the case of a nucleic acid, to a nucleic acid separated from at least one other component (e.g., nucleic acid or polypeptide) that is present with the nucleic acid as found in its natural source and/or that would be present with the nucleic acid when expressed by a cell. A chemically synthesized nucleic acid or one synthesized using in vitro transcription/translation is considered “isolated.”

The term “gene” means a nucleic acid sequence which is transcribed (DNA) to RNA in vitro or in vivo when operably linked to appropriate regulatory sequences. The gene can include regulatory regions preceding and following the coding region, e.g. 5′ untranslated (5′UTR) or “leader” sequences and 3′ UTR or “trailer” sequences, as well as intervening sequences (introns) between individual coding segments (exons).

As used herein, the term “complementary” refers to the ability of nucleotides to form hydrogen-bonded base pairs. In some embodiment, complementary refers to hydrogen-bonded base pair formation preferences between the nucleotide bases G, A, T, C and U, such that when two given polynucleotides or polynucleotide sequences anneal to each other, A pairs with T and G pairs with C in DNA, and G pairs with C and A pairs with U in RNA. As used herein, “substantially complementary” refers to a nucleic acid molecule or portion thereof (e.g. a primer) having at least 90% complementarity over the entire length of the molecule or portion thereof with a second nucleotide sequence, e.g. 90% complementary, 95% complementary, 98% complementary, 99% complementary, or 100% complementary. As used herein, “substantially identical” refers to a nucleic acid molecule or portion thereof having at least 90% identity over the entire length of a the molecule or portion thereof with a second nucleotide sequence, e.g. 90% identity, 95% identity, 98% identity, 99% identity, or 100% identity.

As used herein, “specific” when used in the context of a primer specific for a target nucleic acid refers to a level of complementarity between the primer and the target such that there exists an annealing temperature at which the primer will anneal to and mediate amplification of the target nucleic acid and will not anneal to or mediate amplification of non-target sequences present in a sample.

As used herein, “amplified product”, “amplification product”, or “amplicon” refers to oligonucleotides resulting from an amplification reaction that are copies of a portion of a particular target nucleic acid template strand and/or its complementary sequence, which correspond in nucleotide sequence to the template nucleic acid sequence and/or its complementary sequence. An amplification product can further comprise sequence specific to the primers and which flanks sequence which is a portion of the target nucleic acid and/or its complement. An amplified product, as described herein will generally be double-stranded DNA, although reference can be made to individual strands thereof.

As used herein, a “portion” of a nucleic acid molecule refers to contiguous set of nucleotides comprised by that molecule. A portion can comprise all or only a subset of the nucleotides comprised by the molecule. A portion can be double-stranded or single-stranded.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. lung cancer. The term “treating” includes reducing or alleviating at least one adverse effect or symptom of a condition, disease or disorder associated with a condition. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

The term “statistically significant” or “significantly” refers to statistical significance and generally means a two standard deviation (2SD) below normal, or lower, concentration of the marker.

Other than in the operating examples, or where otherwise indicated, all numbers expressing quantities of ingredients or reaction conditions used herein should be understood as modified in all instances by the term “about.” The term “about” when used in connection with percentages can mean ±1%.

As used herein the term “comprising” or “comprises” is used in reference to compositions, methods, and respective component(s) thereof, that are essential to method or composition, yet open to the inclusion of unspecified elements, whether essential or not.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9). Definitions of common terms in molecular biology can also be found in Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, the present invention was performed using standard procedures, as described, for example in Sambrook et al., Molecular Cloning: A Laboratory Manual (3 ed.), Cold Spring Harbor Laboratory Press, Cold Spring Harbor, N.Y., USA (2001); and Davis et al., Basic Methods in Molecular Biology, Elsevier Science Publishing, Inc., New York, USA (1995) which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, methodologies described in such publications that might be used in connection with technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if appropriate, to employ the compositions, functions and concepts of the above references and application to provide yet further embodiments of the disclosure. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

Technology described herein is further illustrated by the following examples which in no way should be construed as being further limiting.

Some embodiments of the technology described herein can be defined according to any of the following numbered paragraphs:

1. A method of determining the nucleotide sequence contiguous to a known target nucleotide sequence, the method comprising;
(a) hybridizing a target nucleic acid molecule comprising the known target nucleotide sequence with a population of tailed random primers;
(b) extension of a hybridized tailed random primer using the portion of the target nucleic acid molecule downstream of the site of hybridization as a template;
(c) amplifying a portion of the target nucleic acid molecule and the tailed random primer sequence with a first tail primer and a first target-specific primer;
(d) amplifying a portion of the amplicon resulting from step (c) with a second tail primer and a second target-specific primer;
(e) sequencing the amplified portion from step (d) using a first and second sequencing primer;
wherein the population of tailed random primers comprises single-stranded oligonucleotide molecules having a 5′ nucleic acid sequence identical or complementary to a first sequencing primer and a 3′ nucleic acid sequence comprising from about 6 to about 12 random nucleotides;
wherein the first target-specific primer comprises a nucleic acid sequence that can specifically anneal to the known target nucleotide sequence of the target nucleic acid at the annealing temperature;
wherein the second target-specific primer comprises a 3′ portion comprising a nucleic acid sequence that can specifically anneal to a portion of the known target nucleotide sequence comprised by the amplicon resulting from step (c), and a 5′ portion comprising a nucleic acid sequence that is identical to a second sequencing primer and the second target-specific primer is nested with respect to the first target-specific primer;
wherein the first tail primer comprises a nucleic acid sequence identical or complementary to all or a portion of the 5′ portion of the tailed random primer; and
wherein the second tail primer comprises a nucleic acid sequence identical or complementary to a portion of the first sequencing primer and is nested with respect to the first tail primer.

2. The method of paragraph 1, wherein the 5′ nucleic acid sequence of the tailed random primers is identical to a first sequencing primer.

3. The method of any of paragraphs 1-2, wherein the first tail primer comprises a nucleic acid sequence identical to the 5′ portion of the tailed random primer.

4. The method of any of paragraphs 1-3, wherein the second tail primer comprises a nucleic acid sequence identical to a portion of the first sequencing primer.

5. The method of any of paragraphs 1-4, wherein the each tailed random primer further comprises a spacer nucleic acid sequence between the 5′ nucleic acid sequence identical or complementary to a first sequencing primer and the 3′ nucleic acid sequence comprising about 6 to about 12 random nucleotides.

6. The method of any of paragraphs 1-5, wherein the unhybridized primers are removed from the reaction after an extension step.

7. The method of any of paragraphs 1-6, wherein the second tail primer is nested with respect to the first tail primer by at least 3 nucleotides.

8. The method of any of paragraphs 1-7, wherein the first target-specific primer further comprises a 5′ tag sequence portion comprising a nucleic acid sequence of high GC content which is not substantially complementary to or substantially identical to any other portion of any of the primers.

9. The method of any of paragraphs 1-8, wherein the second tail primer is identical to the full-length first sequencing primer.

10. The method of any of paragraphs 1-9, wherein the portions of the target-specific primers that specifically anneal to the known target will anneal specifically at a temperature of about 65° C. in a PCR buffer.

11. The method of any of paragraphs 1-10, wherein the sample comprises genomic DNA.

12. The method of any of paragraphs 1-11, wherein the sample comprises RNA and the method further comprises a first step of subjecting the sample to a reverse transcriptase regimen.

13. The method of any of paragraphs 1-13, wherein the nucleic acids present in the sample have not been subjected to shearing or digestion.

14. The method of any of paragraphs 1-14, wherein the sample comprises single-stranded gDNA or cDNA.

15. The method of any of paragraphs 12-14, wherein the reverse transcriptase regimen comprises the use of random hexamers.

16. The method of any of paragraphs 1-15, wherein a gene rearrangement comprises the known target sequence.

17. The method of paragraph 16, wherein the gene rearrangement is present in a nucleic acid selected from the group consisting of: genomic DNA; RNA; and cDNA.

18. The method of any of paragraphs 16-17, wherein the gene rearrangement comprises an oncogene.

19. The method of paragraph 18, wherein the gene rearrangement comprises a fusion oncogene.

20. The method of any of paragraphs 1-19, wherein the nucleic acid product is sequenced by a next-generation sequencing method.

21. The method of paragraph 20, wherein the next-generation sequencing method comprises a method selected from the group consisting of:
Ion Torrent, Illumina, SOLiD, 454; Massively Parallel Signature Sequencing solid-phase, reversible dye-terminator sequencing; and DNA nanoball sequencing.

22. The method of any of paragraphs 1-21, wherein the first and second sequencing primers are compatible with the selected next-generation sequencing method.

23. The method of any of paragraphs 1-22, wherein the method comprises contacting the sample, or separate portions of the sample, with a plurality of sets of first and second target-specific primers.

24. The method of any of paragraphs 1-23, wherein the method comprises contacting a single reaction mixture comprising the sample with a plurality of sets of first and second target-specific primers.

25. The method of any of paragraphs 1-24, wherein the plurality of sets of first and second target-specific primers specifically anneal to known target nucleotide sequences comprised by separate genes.

26. The method of any of paragraphs 24-25, wherein at least two sets of first and second target-specific primers specifically anneal to different portions of a known target nucleotide sequence.

27. The method of any of paragraphs 24-26, wherein at least two sets of first and second target-specific primers specifically anneal to different portions of a single gene comprising a known target nucleotide sequence.

28. The method of any of paragraphs 24-27, wherein at least two sets of first and second target-specific primers specifically anneal to different exons of a gene comprising a known nucleotide target sequence.

29. The method of any of paragraphs 24-28, wherein the plurality of first target-specific primers comprise identical 5′ tag sequence portions.

30. The method of any of paragraphs 1-29, wherein each amplification step comprises a set of cycles of a PCR amplification regimen from 5 cycles to 20 cycles in length.

31. The method of any of paragraphs 1-30, wherein the target-specific primers and the tail primers are designed such that they will specifically anneal to their complementary sequences at an annealing temperature of from about 61 to 72° C.

32. The method of any of paragraphs 1-31, wherein the target-specific primers and the tail primers are designed such that they will specifically anneal to their complementary sequences at an annealing temperature of about 65° C.

33. The method of any of paragraphs 1-32, wherein the target nucleic acid molecule is from a sample, optionally which is a biological sample obtained from a subject.

34. The method of any of paragraphs 1-33, wherein the sample is obtained from a subject in need of treatment for a disease associated with a genetic alteration.

35. The method of paragraph 34, wherein the disease is cancer.

36. The method of any of paragraphs 1-35, wherein the sample comprises a population of tumor cells.

37. The method of any of paragraphs 1-36, wherein the sample is a tumor biopsy.

38. The method of any of paragraphs 35-37, wherein the cancer is lung cancer.

39. The method of any of paragraphs 1-38, wherein a disease-associated gene comprises the known target sequence.

40. The method of any of paragraphs 1-39, wherein the target nucleic acid is a ribonucleic acid.

41. The method of paragraph 1-39, wherein the target nucleic acid is a deoxyribonucleic acid.

42. The method of paragraph 40, wherein the target nucleic acid is a messenger RNA encoded from a chromosomal segment that comprises a genetic rearrangement.

43. The method of paragraph 41, wherein the target nucleic acid is a chromosomal segment that comprises a portion of a genetic rearrangement.

44. A method of preparing nucleic acids for analysis, the method comprising:
contacting a nucleic acid template comprising with a plurality of different primers that share a common sequence that is 5′ to different hybridization sequences, under conditions to promote template-specific hybridization and extension of at least one of the plurality of different primers;
contacting the extension product of the first step with a first tail primer and a first target-specific primer under conditions to promote template-specific hybridization and extension from the first tail primer and first target-specific primer;
contacting the extension product of the second step with a second tail primer and a second target-specific primer under conditions to promote template-specific hybridization and extension from the second tail primer and second target-specific primer.
wherein the first target-specific primer comprises a nucleic acid sequence that can specifically anneal to a known target nucleotide sequence of the target nucleic acid at the annealing temperature;
wherein the second target-specific primer comprises a 3′ portion comprising a nucleic acid sequence that can specifically anneal to a portion of the known target nucleotide sequence comprised by the amplicon resulting from the second step, and a 5′ portion comprising a nucleic acid sequence that is identical to a second sequencing primer and the second target-specific primer is nested with respect to the first target-specific primer;
wherein the first tail primer comprises a nucleic acid sequence identical or complementary to the common sequence of the primers of the first step; and
wherein the second tail primer comprises a nucleic acid sequence identical or complementary to a portion of the first sequencing primer and is nested with respect to the first tail primer.

45. The method of paragraph 44, wherein the target nucleic acid is a ribonucleic acid.

46. The method of paragraph 44, wherein the target nucleic acid is a deoxyribonucleic acid.

47. The method of paragraph 45, wherein the target nucleic acid is a messenger RNA encoded from a chromosomal segment that comprises a genetic rearrangement.

48. The method of paragraph 46, wherein the target nucleic acid is a chromosomal segment that comprises a portion of a genetic rearrangement.

49. The method of paragraph 48, wherein the genetic rearrangement is an inversion, deletion, or translocation.

50. The method of any one of paragraphs 44-49, further comprising amplifying one or more of the extension products

51. The method of any of paragraphs 44-50, wherein the each of the primers of the first step further comprises a spacer nucleic acid sequence between the common sequence and the hybridization sequence, the spacer sequence comprising about 6 to about 12 random nucleotides.

52. The method of any of paragraphs 44-51, wherein the unhybridized primers are removed from the reaction after extension.

53. The method of any of paragraphs 44-52, wherein the second tail primer is nested with respect to the first tail primer by at least 3 nucleotides.

54. The method of any of paragraphs 44-53, wherein the first target-specific primer further comprises a 5′ tag sequence portion comprising a nucleic acid sequence of high GC content which is not substantially complementary to or substantially identical to any other portion of any of the primers.

55. The method of any of paragraphs 44-54, wherein the portions of the target-specific primers that specifically anneal to the known target will anneal specifically at a temperature of about 65° C. in a PCR buffer.

### EXAMPLES

### Example 1

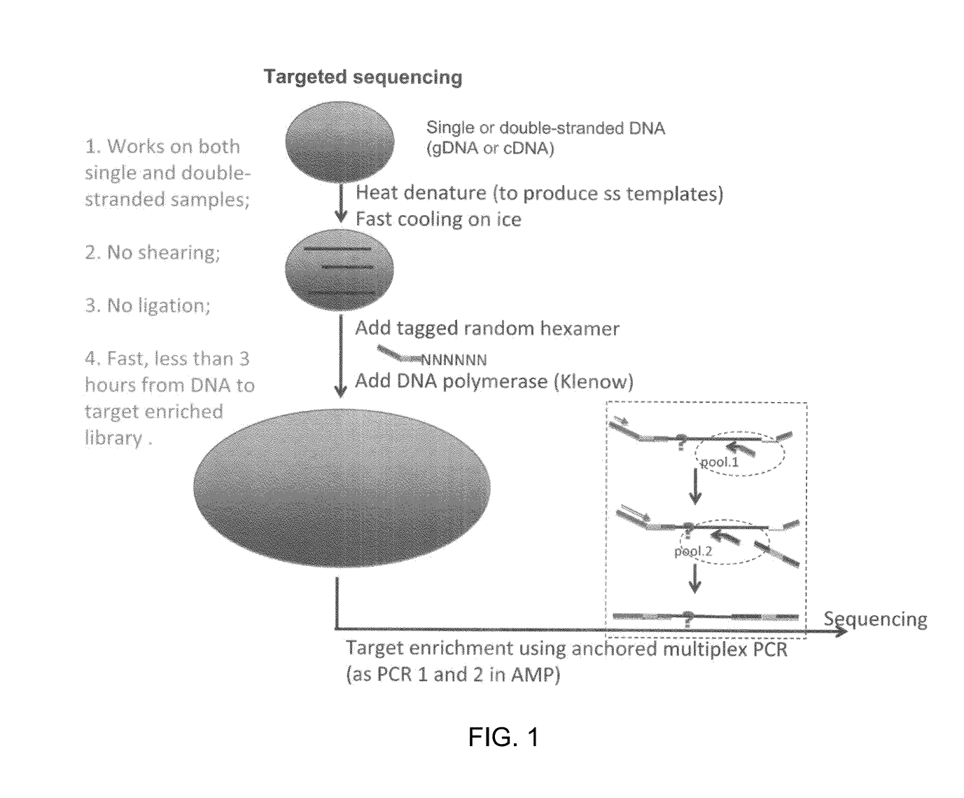

Described herein is a method (described for simplicity as “AMP2” for Anchored Multiplex PCR version 2; see FIG. 1) that comprises an improvement of AMP (Anchored Multiplex PCR) (described, e.g. in U.S. patent application Ser. No. 13/793,564; which is incorporated by reference herein in its entirety). The original AMP is a method to construct targeted sequencing libraries for next generation sequencing (NGS) in which a single type of double-stranded DNA adapter (containing one sequencing primer) is ligated to the double-stranded DNA (gDNA or cDNA) template. Two rounds of hemi-nested PCR are performed with pools of gene specific primers (GSP1s and GSP2s). GSP2 contains the second sequencing primer sequence, thus allowing a fully-competent sequencing library to be completed. Since one side of each and any of multiple fragments has a specific gene specific sequence (the anchor) and the other side has a randomly ligated adaptor, it is termed anchored multiplex PCR.

Described herein is AMP2, which simplifies the approach described above and improves its ability to use poor quality archived nucleic acid which is critical to certain applications (e.g. clinical tumor genotyping). A new synthetic oligonucleotide design for incorporating the first sequencing primer into the library consists of a primer with a 5′ sequencing primer sequence (e.g., an Illumina, Roche, Life Technologies, Ion Torrent or any other NGS method-compatible primer) and a 3′ sequence containing at least 6 random nucleotides (can be up to 12 nucleotides). Step one involves incubation of this oligonucleotide primer with the template DNA (gDNA or cDNA is acceptable), annealing of the oligonucleotide primer randomly with the template, and extension of the primer using a DNA polymerase. Following removal of the unincorporated primers, the new extension products can be used in an amplification protocol similar to that of AMP, starting at the GSP1 PCR step. This new method allows one to avoid mechanical shearing, end-repair, A-tailing, ligation of adapters, and multiple clean-up steps. The AMP2 method also has the advantage of utilizing a random 6 to 12 mer sequencing primer that will be sequenced and could serve as a unique molecular barcode, allowing bioinformatic algorithms to improve variant calling accuracy for both single nucleotide, indel, and copy number variants. Thus AMP2 permits a simplified targeted library construction method with improved performance for nucleic acid from archived material regardless if they are in double-stranded or single-stranded form, and permits higher-quality variant/mutation assessment.

The disclosed method allows for several novel improvements over the previously described “AMP” method:
1. Use of tailed random primer for the 1st step in AMP2 method avoids the need for mechanical shearing of samples nucleic acid needed in AMP1, since the random primed replication of original template will result in sequencing primer-tailed shorter fragments. These fragments are in the required size range for effective downstream NGS library construction.
2. Since shearing is no longer required in AMP2 method, several subsequent steps can be omitted, simplifying and shortening the protocol. Post-shearing end-repair with Klenow enzyme, addition of deoxyadenosine, and ligation of double stranded adapters would not be needed.
3. As previously described, the AMP1 method requires double stranded DNA (gDNA or cDNA) templates for ligation with the double-stranded sequencing adapter. This requirement can limit the effectiveness of the assay with archived samples. Archived samples often have significant degradation of nucleic acids compared to fresh or frozen samples, both typified by fragmentation of the nucleic acid and the presence of a significant fraction of single-stranded nucleic acid. The large amount of single stranded template would prevent effective ligation of double stranded adapters in AMP1. Thus, AMP2 would allow higher library construction success with less starting input material relative to AMP1, which would increase the number of archived samples to be processed and analyzed.

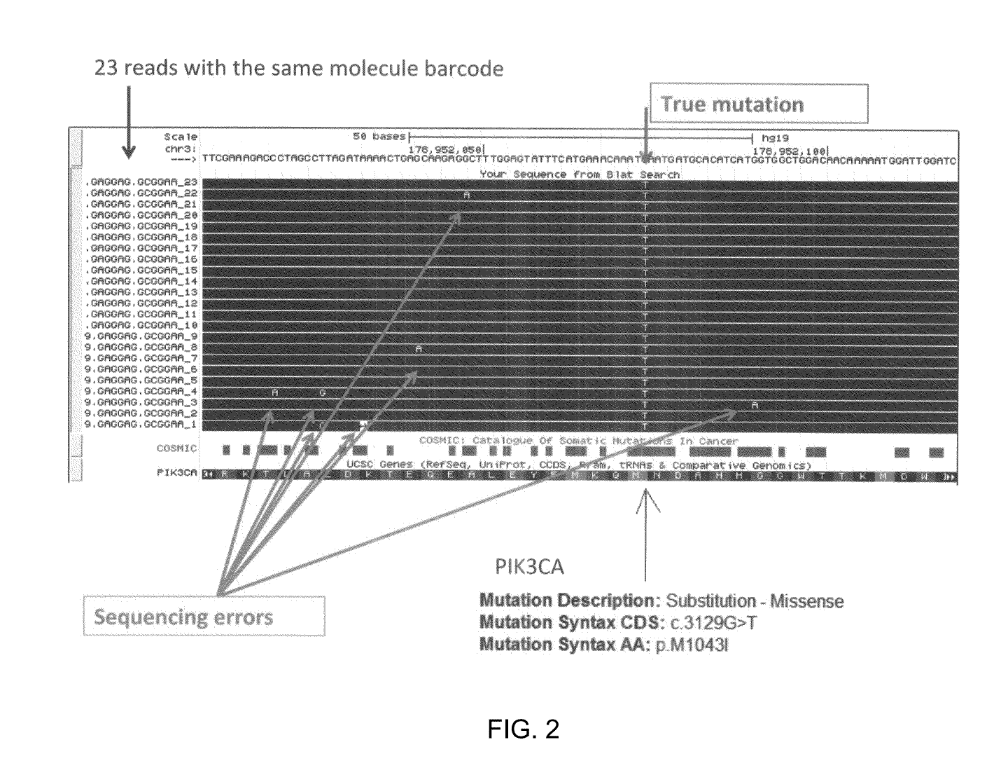

The use of random priming in the first step, creates a unique molecular identifier for each extension reaction, allowing identification (or single molecule barcoding) of each unique template molecule. This offers major advantages, including improved sequencing error correction via bioinformatic algorithms which exploit the molecular barcodes to consolidate truly duplicated sequencing fragments/reads into a singular accurate consensus read (FIG. 2).

The methods described herein permit assays (including diagnostics and companion diagnostics) for the detection of DNA or RNA sequence variants or abundance using next generation sequencing. This can include gene-specific kits, or general-purpose library construction kits suitable for use with the user's targeted primers of choice.

The methods described herein permit the detection of mutations in nucleic acids in targeted sequencing for both germline and somatic tumor mutation applications in humans. This method can also be used for sequencing of non-human nucleic acid.

| SEQ ID NO: 1 | |
|---|---|
| ALK mRNA NCBI Ref Seq: NM_004304 | |
| 1 | agctgcaagt ggcgggcgcc caggcagatg cgatccagcg gctctggggg cggcagcggt |
| 61 | ggtagcagct ggtacctccc gccgcctctg ttcggagggt cgcggggcac cgaggtgctt |
| 121 | tccggccgcc ctctggtcgg ccacccaaag ccgcgggcgc tgatgatggg tgaggagggg |
| 181 | gcggcaagat ttcgggcgcc cctgccctga acgccctcag ctgctgccgc cggggccgct |
| 241 | ccagtgcctg cgaactctga ggagccgagg cgccggtgag agcaaggacg ctgcaaactt |
| 301 | gcgcagcgcg ggggctggga ttcacgccca gaagttcagc aggcagacag tccgaagcct |
| 361 | tcccgcagcg gagagatagc ttgagggtgc gcaagacggc agcctccgcc ctcggttccc |
| 421 | gcccagaccg ggcagaagag cttggaggag ccaaaaggaa cgcaaaaggc ggccaggaca |
| 481 | gcgtgcagca gctgggagcc gccgttctca gccttaaaag ttgcagagat tggaggctgc |
| 541 | cccgagaggg gacagacccc agctccgact gcggggggca ggagaggacg gtacccaact |
| 601 | gccacctccc ttcaaccata gtagttcctc tgtaccgagc gcagcgagct acagacgggg |
| 661 | gcgcggcact cggcgcggag agcgggaggc tcaaggtccc agccagtgag cccagtgtgc |
| 721 | ttgagtgtct ctggactcgc ccctgagctt ccaggtctgt ttcatttaga ctcctgctcg |
| 781 | cctccgtgca gttgggggaa agcaagagac ttgcgcgcac gcacagtcct ctggagatca |
| 841 | ggtggaagga gccgctgggt accaaggact gttcagagcc tcttcccatc tcggggagag |
| 901 | cgaagggtga ggctgggccc ggagagcagt gtaaacggcc tcctccggcg ggatgggagc |
| 961 | catcgggctc ctgtggctcc tgccgctgct gctttccacg gcagctgtgg gctccgggat |
| 1021 | ggggaccggc cagcgcgcgg gctccccagc tgcggggccg ccgctgcagc cccgggagcc |
| 1081 | actcagctac tcgcgcctgc agaggaagag tctggcagtt gacttcgtgg tgccctcgct |
| 1141 | cttccgtgtc tacgcccggg acctactgct gccaccatcc tcctcggagc tgaaggctgg |
| 1201 | caggcccgag gcccgcggct cgctagctct ggactgcgcc ccgctgctca ggttgctggg |
| 1261 | gccggcgccg ggggtctcct ggaccgccgg ttcaccagcc ccggcagagg cccggacgct |
| 1321 | gtccagggtg ctgaagggcg gctccgtgcg caagctccgg cgtgccaagc agttggtgct |
| 1381 | ggagctgggc gaggaggcga tcttggaggg ttgcgtcggg ccccccgggg aggcggctgt |
| 1441 | ggggctgctc cagttcaatc tcagcgagct gttcagttgg tggattcgcc aaggcgaagg |
| 1501 | gcgactgagg atccgcctga tgcccgagaa gaaggcgtcg gaagtgggca gagagggaag |
| 1561 | gctgtccgcg gcaattcgcg cctcccagcc ccgccttctc ttccagatct tcgggactgg |
| 1621 | tcatagctcc ttggaatcac caacaaacat gccttctcct tctcctgatt attttacatg |
| 1681 | gaatctcacc tggataatga aagactcctt ccctttcctg tctcatcgca gccgatatgg |
| 1741 | tctggagtgc agctttgact tcccctgtga gctggagtat tcccctccac tgcatgacct |
| 1801 | caggaaccag agctggtcct ggcgccgcat cccctccgag gaggcctccc agatggactt |
| 1861 | gctggatggg cctggggcag agcgttctaa ggagatgccc agaggctcct ttctccttct |
| 1921 | caacacctca gctgactcca agcacaccat cctgagtccg tggatgagga gcagcagtga |
| 1981 | gcactgcaca ctggccgtct cggtgcacag gcacctgcag ccctctggaa ggtacattgc |
| 2041 | ccagctgctg ccccacaacg aggctgcaag agagatcctc ctgatgccca ctccagggaa |
| 2101 | gcatggttgg acagtgctcc agggaagaat cgggcgtcca gacaacccat ttcgagtggc |
| 2161 | cctggaatac atctccagtg gaaaccgcag cttgtctgca gtggacttct ttgccctgaa |
| 2221 | gaactgcagt gaaggaacat ccccaggctc caagatggcc ctgcagagct ccttcacttg |
| 2281 | ttggaatggg acagtcctcc agcttgggca ggcctgtgac ttccaccagg actgtgccca |
| 2341 | gggagaagat gagagccaga tgtgccggaa actgcctgtg ggtttttact gcaactttga |
| 2401 | agatggcttc tgtggctgga cccaaggcac actgtcaccc cacactcctc aatggcaggt |
| 2461 | caggacccta aaggatgccc ggttccagga ccaccaagac catgctctat tgctcagtac |
| 2521 | cactgatgtc cccgcttctg aaagtgctac agtgaccagt gctacgtttc ctgcaccgat |
| 2581 | caagagctct ccatgtgagc tccgaatgtc ctggctcatt cgtggagtct tgaggggaaa |
| 2641 | cgtgtccttg gtgctagtgg agaacaaaac cgggaaggag caaggcagga tggtctggca |
| 2701 | tgtcgccgcc tatgaaggct tgagcctgtg gcagtggatg gtgttgcctc tcctcgatgt |
| 2761 | gtctgacagg ttctggctgc agatggtcgc atggtgggga caaggatcca gagccatcgt |
| 2821 | ggcttttgac aatatctcca tcagcctgga ctgctacctc accattagcg gagaggacaa |
| 2881 | gatcctgcag aatacagcac ccaaatcaag aaacctgttt gagagaaacc caaacaagga |
| 2941 | gctgaaaccc ggggaaaatt caccaagaca gacccccatc tttgacccta cagttcattg |
| 3001 | gctgttcacc acatgtgggg ccagcgggcc ccatggcccc acccaggcac agtgcaacaa |
| 3061 | cgcctaccag aactccaacc tgagcgtgga ggtggggagc gagggccccc tgaaaggcat |
| 3121 | ccagatctgg aaggtgccag ccaccgacac ctacagcatc tcgggctacg gagctgctgg |
| 3181 | cgggaaaggc gggaagaaca ccatgatgcg gtcccacggc gtgtctgtgc tgggcatctt |
| 3241 | caacctggag aaggatgaca tgctgtacat cctggttggg cagcagggag aggacgcctg |
| 3301 | ccccagtaca aaccagttaa tccagaaagt ctgcattgga gagaacaatg tgatagaaga |
| 3361 | agaaatccgt gtgaacagaa gcgtgcatga gtgggcagga ggcggaggag gagggggtgg |
| 3421 | agccacctac gtatttaaga tgaaggatgg agtgccggtg cccctgatca ttgcagccgg |
| 3481 | aggtggtggc agggcctacg gggccaagac agacacgttc cacccagaga gactggagaa |
| 3541 | taactcctcg gttctagggc taaacggcaa ttccggagcc gcaggtggtg gaggtggctg |
| 3601 | gaatgataac acttccttgc tctgggccgg aaaatctttg caggagggtg ccaccggagg |
| 3661 | acattcctgc ccccaggcca tgaagaagtg ggggtgggag acaagagggg gtttcggagg |
| 3721 | gggtggaggg gggtgctcct caggtggagg aggcggagga tatataggcg gcaatgcagc |
| 3781 | ctcaaacaat gaccccgaaa tggatgggga agatggggtt tccttcatca gtccactggg |
| 3841 | catcctgtac accccagctt taaaagtgat ggaaggccac ggggaagtga atattaagca |
| 3901 | ttatctaaac tgcagtcact gtgaggtaga cgaatgtcac atggaccctg aaagccacaa |
| 3961 | ggtcatctgc ttctgtgacc acgggacggt gctggctgag gatggcgtct cctgcattgt |
| 4021 | gtcacccacc ccggagccac acctgccact ctcgctgatc ctctctgtgg tgacctctgc |
| 4081 | cctcgtggcc gccctggtcc tggctttctc cggcatcatg attgtgtacc gccggaagca |
| 4141 | ccaggagctg caagccatgc agatggagct gcagagccct gagtacaagc tgagcaagct |
| 4201 | ccgcacctcg accatcatga ccgactacaa ccccaactac tgctttgctg gcaagacctc |
| 4261 | ctccatcagt gacctgaagg aggtgccgcg gaaaaacatc accctcattc ggggtctggg |
| 4321 | ccatggcgcc tttggggagg tgtatgaagg ccaggtgtcc ggaatgccca acgacccaag |
| 4381 | ccccctgcaa gtggctgtga agacgctgcc tgaagtgtgc tctgaacagg acgaactgga |
| 4441 | tttcctcatg gaagccctga tcatcagcaa attcaaccac cagaacattg ttcgctgcat |
| 4501 | tggggtgagc ctgcaatccc tgccccggtt catcctgctg gagctcatgg cggggggaga |
| 4561 | cctcaagtcc ttcctccgag agacccgccc tcgcccgagc cagccctcct ccctggccat |
| 4621 | gctggacctt ctgcacgtgg ctcgggacat tgcctgtggc tgtcagtatt tggaggaaaa |
| 4681 | ccacttcatc caccgagaca ttgctgccag aaactgcctc ttgacctgtc caggccctgg |
| 4741 | aagagtggcc aagattggag acttcgggat ggcccgagac atctacaggg cgagctacta |
| 4801 | tagaaaggga ggctgtgcca tgctgccagt taagtggatg cccccagagg ccttcatgga |
| 4861 | aggaatattc acttctaaaa cagacacatg gtcctttgga gtgctgctat gggaaatctt |
| 4921 | ttctcttgga tatatgccat accccagcaa aagcaaccag gaagttctgg agtttgtcac |
| 4981 | cagtggaggc cggatggacc cacccaagaa ctgccctggg cctgtatacc ggataatgac |
| 5041 | tcagtgctgg caacatcagc ctgaagacag gcccaacttt gccatcattt tggagaggat |
| 5101 | tgaatactgc acccaggacc cggatgtaat caacaccgct ttgccgatag aatatggtcc |
| 5161 | acttgtggaa gaggaagaga aagtgcctgt gaggcccaag gaccctgagg gggttcctcc |
| 5221 | tctcctggtc tctcaacagg caaaacggga ggaggagcgc agcccagctg ccccaccacc |
| 5281 | tctgcctacc acctcctctg gcaaggctgc aaagaaaccc acagctgcag agatctctgt |
| 5341 | tcgagtccct agagggccgg ccgtggaagg gggacacgtg aatatggcat tctctcagtc |
| 5401 | caaccctcct tcggagttgc acaaggtcca cggatccaga aacaagccca ccagcttgtg |
| 5461 | gaacccaacg tacggctcct ggtttacaga gaaacccacc aaaaagaata atcctatagc |
| 5521 | aaagaaggag ccacacgaca ggggtaacct ggggctggag ggaagctgta ctgtcccacc |
| 5581 | taacgttgca actgggagac ttccgggggc ctcactgctc ctagagccct cttcgctgac |
| 5641 | tgccaatatg aaggaggtac ctctgttcag gctacgtcac ttcccttgtg ggaatgtcaa |
| 5701 | ttacggctac cagcaacagg gcttgccctt agaagccgct actgcccctg gagctggtca |
| 5761 | ttacgaggat accattctga aaagcaagaa tagcatgaac cagcctgggc cctgagctcg |
| 5821 | gtcgcacact cacttctctt ccttgggatc cctaagaccg tggaggagag agaggcaatg |
| 5881 | gctccttcac aaaccagaga ccaaatgtca cgttttgttt tgtgccaacc tattttgaag |
| 5941 | taccaccaaa aaagctgtat tttgaaaatg ctttagaaag gttttgagca tgggttcatc |
| 6001 | ctattctttc gaaagaagaa aatatcataa aaatgagtga taaatacaag gcccagatgt |
| 6061 | ggttgcataa ggtttttatg catgtttgtt gtatacttcc ttatgcttct ttcaaattgt |
| 6121 | gtgtgctctg cttcaatgta gtcagaatta gctgcttcta tgtttcatag ttggggtcat |
| 6181 | agatgtttcc ttgccttgtt gatgtggaca tgagccattt gaggggagag ggaacggaaa |
| 6241 | taaaggagtt atttgtaatg actaaaa |
| SEQ ID NO: 2 | |
| ROS1 mRNA NCBI Ref Seq: NM_002944 | |
| 1 | caagctttca agcattcaaa ggtctaaatg aaaaaggcta agtattattt caaaaggcaa |
| 61 | gtatatccta atatagcaaa acaaacaaag caaaatccat cagctactcc tccaattgaa |
| 121 | gtgatgaagc ccaaataatt catatagcaa aatggagaaa attagaccgg ccatctaaaa |
| 181 | atctgccatt ggtgaagtga tgaagaacat ttactgtctt attccgaagc ttgtcaattt |
| 241 | tgcaactctt ggctgcctat ggatttctgt ggtgcagtgt acagttttaa atagctgcct |
| 301 | aaagtcgtgt gtaactaatc tgggccagca gcttgacctt ggcacaccac ataatctgag |
| 361 | tgaaccgtgt atccaaggat gtcacttttg gaactctgta gatcagaaaa actgtgcttt |
| 421 | aaagtgtcgg gagtcgtgtg aggttggctg tagcagcgcg gaaggtgcat atgaagagga |
| 481 | agtactggaa aatgcagacc taccaactgc tccctttgct tcttccattg gaagccacaa |
| 541 | tatgacatta cgatggaaat ctgcaaactt ctctggagta aaatacatca ttcagtggaa |
| 601 | atatgcacaa cttctgggaa gctggactta tactaagact gtgtccagac cgtcctatgt |
| 661 | ggtcaagccc ctgcacccct tcactgagta cattttccga gtggtttgga tcttcacagc |
| 721 | gcagctgcag ctctactccc ctccaagtcc cagttacagg actcatcctc atggagttcc |
| 781 | tgaaactgca cctttgatta ggaatattga gagctcaagt cccgacactg tggaagtcag |
| 841 | ctgggatcca cctcaattcc caggtggacc tattttgggt tataacttaa ggctgatcag |
| 901 | caaaaatcaa aaattagatg cagggacaca gagaaccagt ttccagtttt actccacttt |
| 961 | accaaatact atctacaggt tttctattgc agcagtaaat gaagttggtg agggtccaga |
| 1021 | agcagaatct agtattacca cttcatcttc agcagttcaa caagaggaac agtggctctt |
| 1081 | tttatccaga aaaacttctc taagaaagag atctttaaaa catttagtag atgaagcaca |
| 1141 | ttgccttcgg ttggatgcta tataccataa tattacagga atatctgttg atgtccacca |
| 1201 | gcaaattgtt tatttctctg aaggaactct catatgggcg aagaaggctg ccaacatgtc |
| 1261 | tgatgtatct gacctgagaa ttttttacag aggttcagga ttaatttctt ctatctccat |
| 1321 | agattggctt tatcaaagaa tgtatttcat catggatgaa ctggtatgtg tctgtgattt |
| 1381 | agagaactgc tcaaacatcg aggaaattac tccaccctct attagtgcac ctcaaaaaat |
| 1441 | tgtggctgat tcatacaatg ggtatgtctt ttacctcctg agagatggca tttatagagc |
| 1501 | agaccttcct gtaccatctg gccggtgtgc agaagctgtg cgtattgtgg agagttgcac |
| 1561 | gttaaaggac tttgcaatca agccacaagc caagcgaatc atttacttca atgacactgc |
| 1621 | ccaagtcttc atgtcaacat ttctggatgg ctctgcttcc catctcatcc tacctcgcat |
| 1681 | cccctttgct gatgtgaaaa gttttgcttg tgaaaacaat gactttcttg tcacagatgg |
| 1741 | caaggtcatt ttccaacagg atgctttgtc ttttaatgaa ttcatcgtgg gatgtgacct |
| 1801 | gagtcacata gaagaatttg ggtttggtaa cttggtcatc tttggctcat cctcccagct |
| 1861 | gcaccctctg ccaggccgcc cgcaggagct ttcggtgctg tttggctctc accaggctct |
| 1921 | tgttcaatgg aagcctcctg cccttgccat aggagccaat gtcatcctga tcagtgatat |
| 1981 | tattgaactc tttgaattag gcccttctgc ctggcagaac tggacctatg aggtgaaagt |
| 2041 | atccacccaa gaccctcctg aagtcactca tattttcttg aacataagtg gaaccatgct |
| 2101 | gaatgtacct gagctgcaga gtgctatgaa atacaaggtt tctgtgagag caagttctcc |
| 2161 | aaagaggcca ggcccctggt cagagccctc agtgggtact accctggtgc cagctagtga |
| 2221 | accaccattt atcatggctg tgaaagaaga tgggctttgg agtaaaccat taaatagctt |
| 2281 | tggcccagga gagttcttat cctctgatat aggaaatgtg tcagacatgg attggtataa |
| 2341 | caacagcctc tactacagtg acacgaaagg cgacgttttt gtgtggctgc tgaatgggac |
| 2401 | ggatatctca gagaattatc acctacccag cattgcagga gcaggggctt tagcttttga |
| 2461 | gtggctgggt cactttctct actgggctgg aaagacatat gtgatacaaa ggcagtctgt |
| 2521 | gttgacggga cacacagaca ttgttaccca cgtgaagcta ttggtgaatg acatggtggt |
| 2581 | ggattcagtt ggtggatatc tctactggac cacactctat tcagtggaaa gcaccagact |
| 2641 | aaatggggaa agttcccttg tactacagac acagccttgg ttttctggga aaaaggtaat |
| 2701 | tgctctaact ttagacctca gtgatgggct cctgtattgg ttggttcaag acagtcaatg |
| 2761 | tattcacctg tacacagctg ttcttcgggg acagagcact ggggatacca ccatcacaga |
| 2821 | atttgcagcc tggagtactt ctgaaatttc ccagaatgca ctgatgtact atagtggtcg |
| 2881 | gctgttctgg atcaatggct ttaggattat cacaactcaa gaaataggtc agaaaaccag |
| 2941 | tgtctctgtt ttggaaccag ccagatttaa tcagttcaca attattcaga catcccttaa |
| 3001 | gcccctgcca gggaactttt cctttacccc taaggttatt ccagattctg ttcaagagtc |
| 3061 | ttcatttagg attgaaggaa atgcttcaag ttttcaaatc ctgtggaatg gtccccctgc |
| 3121 | ggtagactgg ggtgtagttt tctacagtgt agaatttagt gctcattcta agttcttggc |
| 3181 | tagtgaacaa cactctttac ctgtatttac tgtggaagga ctggaacctt atgccttatt |
| 3241 | taatctttct gtcactcctt atacctactg gggaaagggc cccaaaacat ctctgtcact |
| 3301 | tcgagcacct gaaacagttc catcagcacc agagaacccc agaatattta tattaccaag |
| 3361 | tggaaaatgc tgcaacaaga atgaagttgt ggtggaattt aggtggaaca aacctaagca |
| 3421 | tgaaaatggg gtgttaacaa aatttgaaat tttctacaat atatccaatc aaagtattac |
| 3481 | aaacaaaaca tgtgaagact ggattgctgt caatgtcact ccctcagtga tgtcttttca |
| 3541 | acttgaaggc atgagtccca gatgctttat tgccttccag gttagggcct ttacatctaa |
| 3601 | ggggccagga ccatatgctg acgttgtaaa gtctacaaca tcagaaatca acccatttcc |
| 3661 | tcacctcata actcttcttg gtaacaagat agttttttta gatatggatc aaaatcaagt |
| 3721 | tgtgtggacg ttttcagcag aaagagttat cagtgccgtt tgctacacag ctgataatga |
| 3781 | gatgggatat tatgctgaag gggactcact ctttcttctg cacttgcaca atcgctctag |
| 3841 | ctctgagctt ttccaagatt cactggtttt tgatatcaca gttattacaa ttgactggat |
| 3901 | ttcaaggcac ctctactttg cactgaaaga atcacaaaat ggaatgcaag tatttgatgt |
| 3961 | tgatcttgaa cacaaggtga aatatcccag agaggtgaag attcacaata ggaattcaac |
| 4021 | aataatttct ttttctgtat atcctctttt aagtcgcttg tattggacag aagtttccaa |
| 4081 | ttttggctac cagatgttct actacagtat tatcagtcac accttgcacc gaattctgca |
| 4141 | acccacagct acaaaccaac aaaacaaaag gaatcaatgt tcttgtaatg tgactgaatt |
| 4201 | tgagttaagt ggagcaatgg ctattgatac ctctaaccta gagaaaccat tgatatactt |
| 4261 | tgccaaagca caagagatct gggcaatgga tctggaaggc tgtcagtgtt ggagagttat |
| 4321 | cacagtacct gctatgctcg caggaaaaac ccttgttagc ttaactgtgg atggagatct |
| 4381 | tatatactgg atcatcacag caaaggacag cacacagatt tatcaggcaa agaaaggaaa |
| 4441 | tggggccatc gtttcccagg tgaaggccct aaggagtagg catatcttgg cttacagttc |
| 4501 | agttatgcag ccttttccag ataaagcgtt tctgtctcta gcttcagaca ctgtggaacc |
| 4561 | aactatactt aatgccacta acactagcct cacaatcaga ttacctctgg ccaagacaaa |
| 4621 | cctcacatgg tatggcatca ccagccctac tccaacatac ctggtttatt atgcagaagt |
| 4681 | taatgacagg aaaaacagct ctgacttgaa atatagaatt ctggaatttc aggacagtat |
| 4741 | agctcttatt gaagatttac aaccattttc aacatacatg atacagatag ctgtaaaaaa |
| 4801 | ttattattca gatcctttgg aacatttacc accaggaaaa gagatttggg gaaaaactaa |
| 4861 | aaatggagta ccagaggcag tgcagctcat taatacaact gtgcggtcag acaccagcct |
| 4921 | cattatatct tggagagaat ctcacaagcc aaatggacct aaagaatcag tccgttatca |
| 4981 | gttggcaatc tcacacctgg ccctaattcc tgaaactcct ctaagacaaa gtgaatttcc |
| 5041 | aaatggaagg ctcactctcc ttgttactag actgtctggt ggaaatattt atgtgttaaa |
| 5101 | ggttcttgcc tgccactctg aggaaatgtg gtgtacagag agtcatcctg tcactgtgga |
| 5161 | aatgtttaac acaccagaga aaccttattc cttggttcca gagaacacta gtttgcaatt |
| 5221 | taattggaag gctccattga atgttaacct catcagattt tgggttgagc tacagaagtg |
| 5281 | gaaatacaat gagttttacc atgttaaaac ttcatgcagc caaggtcctg cttatgtctg |
| 5341 | taatatcaca aatctacaac cttatacttc atataatgtc agagtagtgg tggtttataa |
| 5401 | gacgggagaa aatagcacct cacttccaga aagctttaag acaaaagctg gagtcccaaa |
| 5461 | taaaccaggc attcccaaat tactagaagg gagtaaaaat tcaatacagt gggagaaagc |
| 5521 | tgaagataat ggatgtagaa ttacatacta tatccttgag ataagaaaga gcacttcaaa |
| 5581 | taatttacag aaccagaatt taaggtggaa gatgacattt aatggatcct gcagtagtgt |
| 5641 | ttgcacatgg aagtccaaaa acctgaaagg aatatttcag ttcagagtag tagctgcaaa |
| 5701 | taatctaggg tttggtgaat atagtggaat cagtgagaat attatattag ttggagatga |
| 5761 | tttttggata ccagaaacaa gtttcatact tactattata gttggaatat ttctggttgt |
| 5821 | tacaatccca ctgacctttg tctggcatag aagattaaag aatcaaaaaa gtgccaagga |
| 5881 | aggggtgaca gtgcttataa acgaagacaa agagttggct gagctgcgag gtctggcagc |
| 5941 | cggagtaggc ctggctaatg cctgctatgc aatacatact cttccaaccc aagaggagat |
| 6001 | tgaaaatctt cctgccttcc ctcgggaaaa actgactctg cgtctcttgc tgggaagtgg |
| 6061 | agcctttgga gaagtgtatg aaggaacagc agtggacatc ttaggagttg gaagtggaga |
| 6121 | aatcaaagta gcagtgaaga ctttgaagaa gggttccaca gaccaggaga agattgaatt |
| 6181 | cctgaaggag gcacatctga tgagcaaatt taatcatccc aacattctga agcagcttgg |
| 6241 | agtttgtctg ctgaatgaac cccaatacat tatcctggaa ctgatggagg gaggagacct |
| 6301 | tcttacttat ttgcgtaaag cccggatggc aacgttttat ggtcctttac tcaccttggt |
| 6361 | tgaccttgta gacctgtgtg tagatatttc aaaaggctgt gtctacttgg aacggatgca |
| 6421 | tttcattcac agggatctgg cagctagaaa ttgccttgtt tccgtgaaag actataccag |
| 6481 | tccacggata gtgaagattg gagactttgg actcgccaga gacatctata aaaatgatta |
| 6541 | ctatagaaag agaggggaag gcctgctccc agttcggtgg atggctccag aaagtttgat |
| 6601 | ggatggaatc ttcactactc aatctgatgt atggtctttt ggaattctga tttgggagat |
| 6661 | tttaactctt ggtcatcagc cttatccagc tcattccaac cttgatgtgt taaactatgt |
| 6721 | gcaaacagga gggagactgg agccaccaag aaattgtcct gatgatctgt ggaatttaat |
| 6781 | gacccagtgc tgggctcaag aacccgacca aagacctact tttcatagaa ttcaggacca |
| 6841 | acttcagtta ttcagaaatt ttttcttaaa tagcatttat aagtccagag atgaagcaaa |
| 6901 | caacagtgga gtcataaatg aaagctttga aggtgaagat ggcgatgtga tttgtttgaa |
| 6961 | ttcagatgac attatgccag ttgctttaat ggaaacgaag aaccgagaag ggttaaacta |
| 7021 | tatggtactt gctacagaat gtggccaagg tgaagaaaag tctgagggtc ctctaggctc |
| 7081 | ccaggaatct gaatcttgtg gtctgaggaa agaagagaag gaaccacatg cagacaaaga |
| 7141 | tttctgccaa gaaaaacaag tggcttactg cccttctggc aagcctgaag gcctgaacta |
| 7201 | tgcctgtctc actcacagtg gatatggaga tgggtctgat taatagcgtt gtttgggaaa |
| 7261 | tagagagttg agataaacac tctcattcag tagttactga aagaaaactc tgctagaatg |
| 7321 | ataaatgtca tggtggtcta taactccaaa taaacaatgc aacgttcc |
| SEQ ID NO: 3 | |
| RET mRNA NCBI Ref Seq: NM_020630 | |
| 1 | agtcccgcga ccgaagcagg gcgcgcagca gcgctgagtg ccccggaacg tgcgtcgcgc |
| 61 | ccccagtgtc cgtcgcgtcc gccgcgcccc gggcggggat ggggcggcca gactgagcgc |
| 121 | cgcacccgcc atccagaccc gccggcccta gccgcagtcc ctccagccgt ggccccagcg |
| 181 | cgcacgggcg atggcgaagg cgacgtccgg tgccgcgggg ctgcgtctgc tgttgctgct |
| 241 | gctgctgccg ctgctaggca aagtggcatt gggcctctac ttctcgaggg atgcttactg |
| 301 | ggagaagctg tatgtggacc aggcggccgg cacgcccttg ctgtacgtcc atgccctgcg |
| 361 | ggacgcccct gaggaggtgc ccagcttccg cctgggccag catctctacg gcacgtaccg |
| 421 | cacacggctg catgagaaca actggatctg catccaggag gacaccggcc tcctctacct |
| 481 | taaccggagc ctggaccata gctcctggga gaagctcagt gtccgcaacc gcggctttcc |
| 541 | cctgctcacc gtctacctca aggtcttcct gtcacccaca tcccttcgtg agggcgagtg |
| 601 | ccagtggcca ggctgtgccc gcgtatactt ctccttcttc aacacctcct ttccagcctg |
| 661 | cagctccctc aagccccggg agctctgctt cccagagaca aggccctcct tccgcattcg |
| 721 | ggagaaccga cccccaggca ccttccacca gttccgcctg ctgcctgtgc agttcttgtg |
| 781 | ccccaacatc agcgtggcct acaggctcct ggagggtgag ggtctgccct tccgctgcgc |
| 841 | cccggacagc ctggaggtga gcacgcgctg ggccctggac cgcgagcagc gggagaagta |
| 901 | cgagctggtg gccgtgtgca ccgtgcacgc cggcgcgcgc gaggaggtgg tgatggtgcc |
| 961 | cttcccggtg accgtgtacg acgaggacga ctcggcgccc accttccccg cgggcgtcga |
| 1021 | caccgccagc gccgtggtgg agttcaagcg gaaggaggac accgtggtgg ccacgctgcg |
| 1081 | tgtcttcgat gcagacgtgg tacctgcatc aggggagctg gtgaggcggt acacaagcac |
| 1141 | gctgctcccc ggggacacct gggcccagca gaccttccgg gtggaacact ggcccaacga |
| 1201 | gacctcggtc caggccaacg gcagcttcgt gcgggcgacc gtacatgact ataggctggt |
| 1261 | tctcaaccgg aacctctcca tctcggagaa ccgcaccatg cagctggcgg tgctggtcaa |
| 1321 | tgactcagac ttccagggcc caggagcggg cgtectettg ctccacttca acgtgtcggt |
| 1381 | gctgccggtc agcctgcacc tgcccagtac ctactccctc tccgtgagca ggagggctcg |
| 1441 | ccgatttgcc cagatcggga aagtctgtgt ggaaaactgc caggcattca gtggcatcaa |
| 1501 | cgtccagtac aagctgcatt cctctggtgc caactgcagc acgctagggg tggtcacctc |
| 1561 | agccgaggac acctcgggga tcctgtttgt gaatgacacc aaggccctgc ggcggcccaa |
| 1621 | gtgtgccgaa cttcactaca tggtggtggc caccgaccag cagacctcta ggcaggccca |
| 1681 | ggcccagctg cttgtaacag tggaggggtc atatgtggcc gaggaggcgg gctgccccct |
| 1741 | gtcctgtgca gtcagcaaga gacggctgga gtgtgaggag tgtggcggcc tgggctcccc |
| 1801 | aacaggcagg tgtgagtgga ggcaaggaga tggcaaaggg atcaccagga acttctccac |
| 1861 | ctgctctccc agcaccaaga cctgccccga cggccactgc gatgttgtgg agacccaaga |
| 1921 | catcaacatt tgccctcagg actgcctccg gggcagcatt gttgggggac acgagcctgg |
| 1981 | ggagccccgg gggattaaag ctggctatgg cacctgcaac tgcttccctg aggaggagaa |
| 2041 | gtgcttctgc gagcccgaag acatccagga tccactgtgc gacgagctgt gccgcacggt |
| 2101 | gatcgcagcc gctgtcctct tctccttcat cgtctcggtg ctgctgtctg ccttctgcat |
| 2161 | ccactgctac cacaagtttg cccacaagcc acccatctcc tcagctgaga tgaccttccg |
| 2221 | gaggcccgcc caggccttcc cggtcagcta ctcctcttcc ggtgcccgcc ggccctcgct |
| 2281 | ggactccatg gagaaccagg tctccgtgga tgccttcaag atcctggagg atccaaagtg |
| 2341 | ggaattccct cggaagaact tggttcttgg aaaaactcta ggagaaggcg aatttggaaa |
| 2401 | agtggtcaag gcaacggcct tccatctgaa aggcagagca gggtacacca cggtggccgt |
| 2461 | gaagatgctg aaagagaacg cctccccgag tgagcttcga gacctgctgt cagagttcaa |
| 2521 | cgtcctgaag caggtcaacc acccacatgt catcaaattg tatggggcct gcagccagga |
| 2581 | tggcccgctc ctcctcatcg tggagtacgc caaatacggc tccctgcggg gcttcctccg |
| 2641 | cgagagccgc aaagtggggc ctggctacct gggcagtgga ggcagccgca actccagctc |
| 2701 | cctggaccac ccggatgagc gggccctcac catgggcgac ctcatctcat ttgcctggca |
| 2761 | gatctcacag gggatgcagt atctggccga gatgaagctc gttcatcggg acttggcagc |
| 2821 | cagaaacatc ctggtagctg aggggcggaa gatgaagatt tcggatttcg gcttgtcccg |
| 2881 | agatgtttat gaagaggatt cctacgtgaa gaggagccag ggtcggattc cagttaaatg |
| 2941 | gatggcaatt gaatcccttt ttgatcatat ctacaccacg caaagtgatg tatggtcttt |
| 3001 | tggtgtcctg ctgtgggaga tcgtgaccct agggggaaac ccctatcctg ggattcctcc |
| 3061 | tgagcggctc ttcaaccttc tgaagaccgg ccaccggatg gagaggccag acaactgcag |
| 3121 | cgaggagatg taccgcctga tgctgcaatg ctggaagcag gagccggaca aaaggccggt |
| 3181 | gtttgcggac atcagcaaag acctggagaa gatgatggtt aagaggagag actacttgga |
| 3241 | ccttgcggcg tccactccat ctgactccct gatttatgac gacggcctct cagaggagga |
| 3301 | gacaccgctg gtggactgta ataatgcccc cctccctcga gccctccctt ccacatggat |
| 3361 | tgaaaacaaa ctctatggta gaatttccca tgcatttact agattctagc accgctgtcc |
| 3421 | cctctgcact atccttcctc tctgtgatgc tttttaaaaa tgtttctggt ctgaacaaaa |
| 3481 | ccaaagtctg ctctgaacct ttttatttgt aaatgtctga ctttgcatcc agtttacatt |
| 3541 | taggcattat tgcaactatg tttttctaaa aggaagtgaa aataagtgta attaccacat |
| 3601 | tgcccagcaa cttaggatgg tagaggaaaa aacagatcag ggcggaactc tcaggggaga |
| 3661 | ccaagaacag gttgaataag gcgcttctgg ggtgggaatc aagtcatagt acttctactt |
| 3721 | taactaagtg gataaatata caaatctggg gaggtattca gttgagaaag gagccaccag |
| 3781 | caccactcag cctgcactgg gagcacagcc aggttccccc agacccctcc tgggcaggca |
| 3841 | ggtgcctctc agaggccacc cggcactggc gagcagccac tggccaagcc tcagccccag |
| 3901 | tcccagccac atgtcctcca tcaggggtag cgaggttgca ggagctggct ggccctggga |
| 3961 | ggacgcaccc ccactgctgt tttcacatcc tttcccttac ccaccttcag gacggttgtc |
| 4021 | acttatgaag tcagtgctaa agctggagca gttgcttttt gaaagaacat ggtctgtggt |
| 4081 | gctgtggtct tacaatggac agtaaatatg gttcttgcca aaactccttc ttttgtcttt |
| 4141 | gattaaatac tagaaattta aaaaaaaaaa aaaa |
| SEQ ID NO: 4 | |
| RET mRNA NCBI Ref Seq: NM_020975 | |
| 1 | agtcccgcga ccgaagcagg gcgcgcagca gcgctgagtg ccccggaacg tgcgtcgcgc |
| 61 | ccccagtgtc cgtcgcgtcc gccgcgcccc gggcggggat ggggcggcca gactgagcgc |
| 121 | cgcacccgcc atccagaccc gccggcccta gccgcagtcc ctccagccgt ggccccagcg |
| 181 | cgcacgggcg atggcgaagg cgacgtccgg tgccgcgggg ctgcgtctgc tgttgctgct |
| 241 | gctgctgccg ctgctaggca aagtggcatt gggcctctac ttctcgaggg atgcttactg |
| 301 | ggagaagctg tatgtggacc aggcggccgg cacgcccttg ctgtacgtcc atgccctgcg |
| 361 | ggacgcccct gaggaggtgc ccagcttccg cctgggccag catctctacg gcacgtaccg |
| 421 | cacacggctg catgagaaca actggatctg catccaggag gacaccggcc tcctctacct |
| 481 | taaccggagc ctggaccata gctcctggga gaagctcagt gtccgcaacc gcggctttcc |
| 541 | cctgctcacc gtctacctca aggtcttcct gtcacccaca tcccttcgtg agggcgagtg |
| 601 | ccagtggcca ggctgtgccc gcgtatactt ctccttcttc aacacctcct ttccagcctg |
| 661 | cagctccctc aagccccggg agctctgctt cccagagaca aggccctcct tccgcattcg |
| 721 | ggagaaccga cccccaggca ccttccacca gttccgcctg ctgcctgtgc agttcttgtg |
| 781 | ccccaacatc agcgtggcct acaggctcct ggagggtgag ggtctgccct tccgctgcgc |
| 841 | cccggacagc ctggaggtga gcacgcgctg ggccctggac cgcgagcagc gggagaagta |
| 901 | cgagctggtg gccgtgtgca ccgtgcacgc cggcgcgcgc gaggaggtgg tgatggtgcc |
| 961 | cttcccggtg accgtgtacg acgaggacga ctcggcgccc accttccccg cgggcgtcga |
| 1021 | caccgccagc gccgtggtgg agttcaagcg gaaggaggac accgtggtgg ccacgctgcg |
| 1081 | tgtcttcgat gcagacgtgg tacctgcatc aggggagctg gtgaggcggt acacaagcac |
| 1141 | gctgctcccc ggggacacct gggcccagca gaccttccgg gtggaacact ggcccaacga |
| 1201 | gacctcggtc caggccaacg gcagcttcgt gcgggcgacc gtacatgact ataggctggt |
| 1261 | tctcaaccgg aacctctcca tctcggagaa ccgcaccatg cagctggcgg tgctggtcaa |
| 1321 | tgactcagac ttccagggcc caggagcggg cgtcctcttg ctccacttca acgtgtcggt |
| 1381 | gctgccggtc agcctgcacc tgcccagtac ctactccctc tccgtgagca ggagggctcg |
| 1441 | ccgatttgcc cagatcggga aagtctgtgt ggaaaactgc caggcattca gtggcatcaa |
| 1501 | cgtccagtac aagctgcatt cctctggtgc caactgcagc acgctagggg tggtcacctc |
| 1561 | agccgaggac acctcgggga tcctgtttgt gaatgacacc aaggccctgc ggcggcccaa |
| 1621 | gtgtgccgaa cttcactaca tggtggtggc caccgaccag cagacctcta ggcaggccca |
| 1681 | ggcccagctg cttgtaacag tggaggggtc atatgtggcc gaggaggcgg gctgccccct |
| 1741 | gtcctgtgca gtcagcaaga gacggctgga gtgtgaggag tgtggcggcc tgggctcccc |
| 1801 | aacaggcagg tgtgagtgga ggcaaggaga tggcaaaggg atcaccagga acttctccac |
| 1861 | ctgctctccc agcaccaaga cctgccccga cggccactgc gatgttgtgg agacccaaga |
| 1921 | catcaacatt tgccctcagg actgcctccg gggcagcatt gttgggggac acgagcctgg |
| 1981 | ggagccccgg gggattaaag ctggctatgg cacctgcaac tgcttccctg aggaggagaa |
| 2041 | gtgcttctgc gagcccgaag acatccagga tccactgtgc gacgagctgt gccgcacggt |
| 2101 | gatcgcagcc gctgtcctct tctccttcat cgtctcggtg ctgctgtctg ccttctgcat |
| 2161 | ccactgctac cacaagtttg cccacaagcc acccatctcc tcagctgaga tgaccttccg |
| 2221 | gaggcccgcc caggccttcc cggtcagcta ctcctcttcc ggtgcccgcc ggccctcgct |
| 2281 | ggactccatg gagaaccagg tctccgtgga tgccttcaag atcctggagg atccaaagtg |
| 2341 | ggaattccct cggaagaact tggttcttgg aaaaactcta ggagaaggcg aatttggaaa |
| 2401 | agtggtcaag gcaacggcct tccatctgaa aggcagagca gggtacacca cggtggccgt |
| 2461 | gaagatgctg aaagagaacg cctccccgag tgagcttcga gacctgctgt cagagttcaa |
| 2521 | cgtcctgaag caggtcaacc acccacatgt catcaaattg tatggggcct gcagccagga |
| 2581 | tggcccgctc ctcctcatcg tggagtacgc caaatacggc tccctgcggg gcttcctccg |
| 2641 | cgagagccgc aaagtggggc ctggctacct gggcagtgga ggcagccgca actccagctc |
| 2701 | cctggaccac ccggatgagc gggccctcac catgggcgac ctcatctcat ttgcctggca |
| 2761 | gatctcacag gggatgcagt atctggccga gatgaagctc gttcatcggg acttggcagc |
| 2821 | cagaaacatc ctggtagctg aggggcggaa gatgaagatt tcggatttcg gcttgtcccg |
| 2881 | agatgtttat gaagaggatt cctacgtgaa gaggagccag ggtcggattc cagttaaatg |
| 2941 | gatggcaatt gaatcccttt ttgatcatat ctacaccacg caaagtgatg tatggtcttt |
| 3001 | tggtgtcctg ctgtgggaga tcgtgaccct agggggaaac ccctatcctg ggattcctcc |
| 3061 | tgagcggctc ttcaaccttc tgaagaccgg ccaccggatg gagaggccag acaactgcag |
| 3121 | cgaggagatg taccgcctga tgctgcaatg ctggaagcag gagccggaca aaaggccggt |
| 3181 | gtttgcggac atcagcaaag acctggagaa gatgatggtt aagaggagag actacttgga |
| 3241 | ccttgcggcg tccactccat ctgactccct gatttatgac gacggcctct cagaggagga |
| 3301 | gacaccgctg gtggactgta ataatgcccc cctccctcga gccctccctt ccacatggat |
| 3361 | tgaaaacaaa ctctatggca tgtcagaccc gaactggcct ggagagagtc ctgtaccact |
| 3421 | cacgagagct gatggcacta acactgggtt tccaagatat ccaaatgata gtgtatatgc |
| 3481 | taactggatg ctttcaccct cagcggcaaa attaatggac acgtttgata gttaacattt |
| 3541 | ctttgtgaaa ggtaatggac tcacaagggg aagaaacatg ctgagaatgg aaagtctacc |
| 3601 | ggccctttct ttgtgaacgt cacattggcc gagccgtgtt cagttcccag gtggcagact |
| 3661 | cgtttttggt agtttgtttt aacttccaag gtggttttac ttctgatagc cggtgatttt |
| 3721 | ccctcctagc agacatgcca caccgggtaa gagctctgag tcttagtggt taagcattcc |
| 3781 | tttctcttca gtgcccagca gcacccagtg ttggtctgtg tccatcagtg accaccaaca |
| 3841 | ttctgtgttc acatgtgtgg gtccaacact tactacctgg tgtatgaaat tggacctgaa |
| 3901 | ctgttggatt tttctagttg ccgccaaaca aggcaaaaaa atttaaacat gaagcacaca |
| 3961 | cacaaaaaag gcagtaggaa aaatgctggc cctgatgacc tgtccttatt cagaatgaga |
| 4021 | gactgcgggg ggggcctggg ggtagtgtca atgcccctcc agggctggag gggaagaggg |
| 4081 | gccccgagga tgggcctggg ctcagcattc gagatcttga gaatgatttt tttttaatca |
| 4141 | tgcaaccttt ccttaggaag acatttggtt ttcatcatga ttaagatgat tcctagattt |
| 4201 | agcacaatgg agagattcca tgccatcttt actatgtgga tggtggtatc agggaagagg |
| 4261 | gctcacaaga cacatttgtc ccccgggccc accacatcat cctcacgtgt tcggtactga |
| 4321 | gcagccacta cccctgatga gaacagtatg aagaaagggg gctgttggag tcccagaatt |
| 4381 | gctgacagca gaggctttgc tgctgtgaat cccacctgcc accagcctgc agcacacccc |
| 4441 | acagccaagt agaggcgaaa gcagtggctc atcctacctg ttaggagcag gtagggcttg |
| 4501 | tactcacttt aatttgaatc ttatcaactt actcataaag ggacaggcta gctagctgtg |
| 4561 | ttagaagtag caatgacaat gaccaaggac tgctacacct ctgattacaa ttctgatgtg |
| 4621 | aaaaagatgg tgtttggctc ttatagagcc tgtgtgaaag gcccatggat cagctcttcc |
| 4681 | tgtgtttgta atttaatgct gctacaagat gtttctgttt cttagattct gaccatgact |
| 4741 | cataagcttc ttgtcattct tcattgcttg tttgtggtca cagatgcaca acactcctcc |
| 4801 | agtcttgtgg gggcagcttt tgggaagtct cagcagctct tctggctgtg ttgtcagcac |
| 4861 | tgtaacttcg cagaaaagag tcggattacc aaaacactgc ctgctcttca gacttaaagc |
| 4921 | actgatagga cttaaaatag tctcattcaa atactgtatt ttatataggc atttcacaaa |
| 4981 | aacagcaaaa ttgtggcatt ttgtgaggcc aaggcttgga tgcgtgtgta atagagcctt |
| 5041 | gtggtgtgtg cgcacacacc cagagggaga gtttgaaaaa tgcttattgg acacgtaacc |
| 5101 | tggctctaat ttgggctgtt tttcagatac actgtgataa gttcttttac aaatatctat |
| 5161 | agacatggta aacttttggt tttcagatat gcttaatgat agtcttacta aatgcagaaa |
| 5221 | taagaataaa ctttctcaaa ttattaaaaa tgcctacaca gtaagtgtga attgctgcaa |
| 5281 | caggtttgtt ctcaggaggg taagaactcc aggtctaaac agctgaccca gtgatgggga |
| 5341 | atttatcctt gaccaattta tccttgacca ataacctaat tgtctattcc tgagttataa |
| 5401 | aagtccccat ccttattagc tctactggaa ttttcataca cgtaaatgca gaagttacta |
| 5461 | agtattaagt attactgagt attaagtagt aatctgtcag ttattaaaat ttgtaaaatc |
| 5521 | tatttatgaa aggtcattaa accagatcat gttccttttt ttgtaatcaa ggtgactaag |
| 5581 | aaaatcagtt gtgtaaataa aatcatgtat cataaaaaaa aaaaaaaaa |

What is claimed herein is:

1. A method of determining the nucleotide sequence contiguous to a known target nucleotide sequence, the method comprising;
(a) hybridizing a target nucleic acid molecule comprising the known target nucleotide sequence with a population of tailed random primers;
(b) extension of a hybridized tailed random primer using the portion of the target nucleic acid molecule downstream of the site of hybridization as a template;
(c) amplifying a portion of the target nucleic acid molecule and the tailed random primer sequence with a first tail primer and a first target-specific primer;
(d) amplifying a portion of the amplicon resulting from step (c) with a second tail primer and a second target-specific primer;
(e) sequencing the amplified portion from step (d) using a first and second sequencing primer;
wherein the population of tailed random primers comprises single-stranded oligonucleotide molecules having a 5′ nucleic acid sequence identical or complementary to a first sequencing primer and a 3′ nucleic acid sequence comprising from about 6 to about 12 random nucleotides;
wherein the first target-specific primer comprises a nucleic acid sequence that can specifically anneal to the known target nucleotide sequence of the target nucleic acid at the annealing temperature;
wherein the second target-specific primer comprises a 3′ portion comprising a nucleic acid sequence that can specifically anneal to a portion of the known target nucleotide sequence comprised by the amplicon resulting from step (c), and a 5′ portion comprising a nucleic acid sequence that is identical to a second sequencing primer and the second target-specific primer is nested with respect to the first target-specific primer;
wherein the first tail primer comprises a nucleic acid sequence identical or complementary to all or a portion of the 5′ portion of the tailed random primer; and
wherein the second tail primer comprises a nucleic acid sequence identical or complementary to a portion of the first sequencing primer and is nested with respect to the first tail primer.

2. The method of claim 1, wherein the 5′ nucleic acid sequence of the tailed random primers is identical to a first sequencing primer.

3. The method of claim 1, wherein the first tail primer comprises a nucleic acid sequence identical to the 5′ portion of the tailed random primer.

4. The method of claim 1, wherein the second tail primer comprises a nucleic acid sequence identical to a portion of the first sequencing primer.

5. The method of claim 1, wherein the each tailed random primer further comprises a spacer nucleic acid sequence between the 5′ nucleic acid sequence identical or complementary to a first sequencing primer and the 3′ nucleic acid sequence comprising about 6 to about 12 random nucleotides.

6. The method of claim 1, wherein the unhybridized primers are removed from the reaction after an extension step.

7. The method of claim 1, wherein the second tail primer is nested with respect to the first tail primer by at least 3 nucleotides.

8. The method of claim 1, wherein the first target-specific primer further comprises a 5′ tag sequence portion comprising a nucleic acid sequence of high GC content which is not substantially complementary to or substantially identical to any other portion of any of the primers.

9. The method of claim 1, wherein the second tail primer is identical to the full-length first sequencing primer.

10. The method of claim 1, wherein the portions of the target-specific primers that specifically anneal to the known target will anneal specifically at a temperature of about 65° C. in a PCR buffer.

11. The method of claim 1, wherein the sample comprises genomic DNA.

12. The method of claim 1, wherein the sample comprises RNA and the method further comprises a first step of subjecting the sample to a reverse transcriptase regimen.

13. The method of claim 1, wherein the nucleic acids present in the sample have not been subjected to shearing or digestion.

14. The method of claim 1, wherein the sample comprises single-stranded gDNA or cDNA.

15. The method of claim 12, wherein the reverse transcriptase regimen comprises the use of random hexamers.

16. The method of claim 1, wherein a gene rearrangement comprises the known target sequence.

17. The method of claim 16, wherein the gene rearrangement is present in a nucleic acid selected from the group consisting of: genomic DNA; RNA; and cDNA.

18. The method of claim 16, wherein the gene rearrangement comprises an oncogene.

19. The method of claim 18, wherein the gene rearrangement comprises a fusion oncogene.

20. The method of claim 1, wherein the nucleic acid product is sequenced by a next-generation sequencing method.
